(19) United States
(12) Patent Application Publication
(10) Pub. No.: US 2024/0285831 A1
(43) Pub. Date: Aug. 29, 2024

(54) METHODS OF USE OF MODULATORS TO IMPROVE NERVE REGENERATION

(71) Applicants: Edward KEEFER, Dallas, TX (US); The Board of Regents of The University of Texas System, Austin, TX (US)

(72) Inventors: Jonathan CHENG, Dallas, TX (US); Edward KEEFER, Dallas, TX (US); Srikanth VASUDEVAN, Dallas, TX (US)

(73) Assignees: Edward KEEFER, Dallas, TX (US); The Board of Regents of The University of Texas System, Austin, TX (US)

(21) Appl. No.: 18/649,976

(22) Filed: Apr. 29, 2024

Related U.S. Application Data
(63) Continuation-in-part of application No. 16/922,355, filed on Jul. 7, 2020, now Pat. No. 11,969,522.
(60) Provisional application No. 62/871,552, filed on Jul. 8, 2019.

(51) Int. Cl. A61L 27/54; A61K 31/5355; A61K 38/20; A61L 27/24

(52) U.S. Cl. CPC A61L 27/54; A61K 31/5355; A61K 38/204; A61L 27/24; A61L 2300/204; A61L 2300/252; A61L 2430/32

(57) ABSTRACT

The present disclosure describes the use of immune modulators to promote nerve growth and regeneration, particularly in the context of nerve deficit stemming from trauma and disease. In particular, the disclosure provides for the use of CXCR4 antagonists, STAT3 activators, and an agent that increases levels of nitric oxide, either alone or in any combination of these drugs, in surgery performed to treat nerve deficit conditions, especially peripheral nerve deficit conditions caused by cut injury or tear injury, the method especially useful in bridging nerve gaps of 3 cm or longer.

### CROSS-REFERENCE TO RELATED APPLICATIONS

This Continuation-in-Part Application is based on, claims priority to, and incorporates by reference in their entirety for all purposes, U.S. Provisional Application Ser. No. 62/871,552, filed Jul. 8, 2019, and U.S. Nonprovisional application Ser. No. 16/922,355, filed Jul. 7, 2020. Both of said applications are entitled “USE OF IMMUNE MODULATORS TO IMPROVE NERVE REGENERATION”.

### FIELD

The present disclosure relates generally to the fields of medicine and neurobiology. More particularly, it concerns compositions and methods, including surgical intervention methods, for the treatment of nerve deficits and/or nerve damage caused by trauma, pathological disease, infection, an autoimmune disease, congenital disorders, or birth defects due to genetic factors. Specifically, the present disclosure relates to the use of CXCR4 antagonists, STAT3 activators, and nitric oxide-increasing agents, either alone as monotherapies, or in fixed ratio or variable ratio dose combinations, to treat these conditions.

### BACKGROUND

Peripheral nerve injuries that require surgical intervention account for an estimated 550,000 patient-cases each year in the United States alone. Magellan Medical Technology Consultants, Inc., MN. This enormous clinical need drives the medical and scientific research and development of peripheral nerve regeneration surgical interventions and surgically imposed injections and implantations. (Yannas et al., (2007). The peripheral nervous system (PNS) has an inherent capacity to regenerate to a certain extent when subjected to injury, while the central nervous system can reorganize, through the process of plasticity in response to an injury. Several commercially available products provide nerve regeneration using exogenous materials to bridge short nerve defects of less than 2-3 cm in length. Lengths greater than this range are known as the “long gap” in neurosurgery. To date, no widely accepted clinical solutions have: surpassed the “long gap” distance for manufactured constructs to bridge nerve gaps longer than 2-3 cm in length, increased the rate of nerve regeneration, nor increased the quality of nerve regeneration. As such, there is a need for finding new methods of treating longer length nerve injury that can satisfy these high standards and will greatly enhance the ability to treat patients suffering from this common and devastating family of conditions.

### SUMMARY

Thus, in accordance with the present disclosure, there is provided a method of increasing peripheral nerve growth, regrowth, or regeneration, by bridging across an empty critical gap in at least one nerve in a subject in need thereof, which may be a medical (human) or veterinary (mammalian, avian, or reptilian) subject, comprising administering to said subject singly, or in combination, a CXCR4 antagonist, a STAT3 activator, and/or an agent that increases nitric oxide content. More specifically, the CXCR4 antagonist is selected from the group consisting of Plerixafor, BL-8040, or WZ 811; the STAT3 activator is selected from the group consisting of colivelin, neuroprotective peptide, ruxolitinib phosphate, a JAK1/JAK2 inhibitor, or IL-6; and the agent that increases nitric oxide content is selected from the group consisting of (+/−)-S-Nitroso-N-acetylpenicillamine, Molsidomine, 3-Morpholinosydnonimine, Hydroxyguanidine sulfate, Tetrahydrobiopterin (THB) dihydrochloride, S-Nitrosoglutathione (GSNO), Streptozotocin (U-9889), Nicorandil, Dephostatin, DETA NONOate, NOC-12, NOC-18, NOC-5, NOC-7, MAHMA NONOate, PAPA NONOate, Sulfo-NONOate disodium salt, Angeliprimes salt, Diethylamine NONOate, NOR-1, NOR-2, NOR-3, NOR-4, Spermine NONOate, beta-Gal NONOate, BNN3, GEA 3162, GEA 5024, Sodium nitroprusside dihydrate, 10-Nitrooleate, BEC, NO-Indomethacin, Pilotyprimes Acid, SE 175, V-PYRRO/NO, Vinyl-L-NIO Hydrochloride, AMI-1, sodium salt, DAF-FM DA (cell permeable), GEA 5583, N-Acetyl-D,L-penicillamine disulfide, SIN-1A/gammaCD Complex, 4-Phenyl-3-furoxancarbonitrile, JS-K, Lansoprazole Sulfone N-Oxide, NO-Aspirin 1, Glyco-SNAP-2, N,N-Dicarboxymethyl-N,N-dinitroso-p-phenylenediamine (Disodium Salt), (2S)-(+)-Amino-6-iodoacetamidohexanoic acid, 4AF DA, BEC ammonium salt, DAF-2 DA (cell permeable), DAN-1 EE hydrochloride, DD1, DD2, Diethylamine NONOate/AM, Fructose-SNAP-1, Glyco-SNAP-1, Guanylyl Cyclase, Hydroxyguanidine hemisulfate, N-Cyclopropyl-N′-hydroxyguanidine hydrochloride, NOR-5, PROLI NONOate, S-Nitrosocaptopril, 4-(p-methoxyphenyl)-1,3,2-Oxathiazolylium-5-olate, 4-chloro-4-phenyl-1,3,2-Oxathiozolylium-5-olate, 4-phenyl-1,3,2-Oxathiazolylium-5-olate, 4-trifluoro-4-phenyl-1,3,2-Oxathiazolylium-5-olate, Tricarbonyldichloro-ruthenium (II) dimer, DL-alpha-Difluoromethylornithine hydrochloride, Geranylgeranylacetone, N-Nitrosodiethylamine, L-NMMA (citrate), or 3-(Methylnitrosamino)propionitrile. SIN-1 chloride, L-Arginine, SNAP, L-arginine or a PDE5 inhibitor, comprising sildenafil or tadalafil, as well as nitroglycerin, isosorbide mononitrate, and isosorbide. The practice of this disclosure may result in bridging critical gaps in nerves where such gaps exist in lengths ranging from at least 3 cm, and up to 6 cm.

The CXCR4 antagonist of choice may be administered alone, or a CXCR4 antagonist may be co-administered in combination with a STAT3 activator, or a CXCR4 antagonist may be administered with an agent that increases nitric oxide content, or a CXCR4 antagonist with a STAT3 activator and an agent that increases nitric oxide content, all in either variable dosage combination ratios, or in fixed dose combination formulations. In a preferred alternative embodiment of the disclosure, a practitioner may optionally take the additional step of inserting a physical support structure into the critical gap. This physical support structure may be composed of a variety of materials selected from the group consisting of poly-lactide acid, polyurethane, polydioxanone, silicone, cellulose, collagen, PLGA, polycaprolactone or processed natural extracellular matrix. In another preferred embodiment of the disclosure, the patient may optionally be further treated by co-administering one or more nerve growth factors, which may advantageously be selected from the group consisting of (a) a neurotrophic growth factor selected from the group consisting of nerve growth factor, brain-derived neurotrophic factor, or neurotrophin-3; (b) a glial-derived growth factor; (c) a pleotropic nerve growth factor; or (d) a vascular endothelial growth factor. The CXCR4 antagonist, the STAT3 activator, and the agent that increases nitric oxide content, and/or said one or more nerve growth factors, may alternatively be independently delivered in a time-dependent release fashion. The CXCR4 antagonist may be administered prior to both the STAT3 activator or the agent that increases nitric oxide content; the CXCR4 antagonist may be administered after both the STAT3 activator or the agent that increases nitric oxide content; or the CXCR4 antagonist may be administered between the STAT3 activator and the agent that increases nitric oxide content.

The method of the present disclosure may treat a variety of damaged nerve disorders, including when the subject suffers from a peripheral nervous system deficit that may be congenital, may be due to trauma or an iatrogenic event, or may be due to cancer, an autoimmune disease, or to an infection. On occasion, the patient may be suffering from a central nervous system deficit in the brain or spinal cord that is causing a physical discontinuity in a nerve or nerve fiber, and this causation may include an abnormality in a cranial nerve or a spinal nerve. Spinal nerve deficits themselves may be caused by a trauma, an iatrogenic event, a congenital disorder, a genetic disorder, an infectious agent, an autoimmune disease, or by cervical, lumbosacral, or thoracic deficits. And, as with any neurological deficit, physical therapy or nerve deficit therapy may be of benefit to the patient before or after utilizing the intervention of the present disclosure. The method of the disclosure may result in improved sensory function, including where the sensory function may include a nociceptive function, or a mechanoceptive function. Motor control deficit may benefit from the use of the disclosure, including for motor control, fine motor control, gross motor control, or autonomic nerve control. This novel method may be useful in initiating and supporting nerve growth along the axonal axis of one or more neuronal cells, initiating or restarting nerve regrowth along the axonal axis of one or more neuronal cells, or supporting neuronal nerve cell regeneration along the axonal axis of one or more neuronal cells, in a subject in need thereof due to traumatic injury, the progression of pathological disease, a congenital disorder, or due to a genetic disorder, in which the novel method of the present disclosure constitutes an effective treatment by initiating the step of administering to said subject one or more compositions of matter selected from the genus of CXCR4 antagonists, one or more compositions of matter selected from the genus of STAT3 activators, and/or one or more compositions of matter selected from the genus of compositions that increases nitric oxide content in cells. The common factor that is present in the wide array of traumas and diseases described above will have the same feature of a nerve or nerve bundle that has become cut, torn, or otherwise physically degraded to the point that there is a break and a gap of some length in the axis of the axon of that nerve cell or nerve bundle, meaning that it is now discontinuous and interrupted and the nerve's circuit that carries electrical signals is open. Thus, the treatment of other categories of nerve damage, for example contusion, are not within the indications of the present disclosure. The method of the present disclosure may result in bridging a critical length gap in a nerve cell, a nerve, or a neuronal population, of at least 3 cm, including for example lengths of 3 cm, 3.5 cm, 4 cm, 4.5 cm, 5 cm, or up to 6 cm. Administration of such compositions of matter preferably comprises the step of administering one or more CXCR4 antagonists alone, or co-administering one or more CXCR4 antagonists with one or more STAT3 activators, or of co-administering one or more CXCR4 antagonists with one or more of a composition of matter whose mechanism of action is that of increasing nitric oxide content, or co-administering one or more CXCR4 antagonists with one or more STAT3 activators and one or more agents that increases nitric oxide content.

A preferred alternative embodiment of the present disclosure may further comprise inserting a physical support structure, such as a tube or a spongiform matrix into the critical length gap, such as for example a structure comprised of poly-lactide acid, polyurethane, polydioxanone, silicone, cellulose, collagen, poly(lactic-co-glycolic acid) (PLGA), polycaprolactone or a processed natural extracellular matrix. The method may further comprise administering to said subject one or more nerve growth factors, preferably such as a neurotrophic growth factor (for example nerve growth factor (NGF), brain-derived neurotrophic factor (BDNG), or neurotrophin-3 (NT-3), a glial-derived nerve growth factor (GDNF), a pleotropic nerve growth factor (PTN), and/or a vascular endothelial growth factor (VEGF) type of nerve growth factor. Any one or more of the CXCR4 antagonist, the STAT3 activator, or the agent that increases nitric oxide content, and/or said one or more nerve growth factors, may optionally be delivered in a time-dependent release fashion.

In one alternative preferred embodiment of the present disclosure, one or more of said CXCR4 antagonist, said STAT3 activator and/or said nitric oxide level enhancer is or are administered or implanted into the area of nerve deficit but optionally with no physical support structure being inserted into said subject patient, and/or optionally with there being no growth factor administered to said subject patient. In another alternative preferred embodiment, the CXCR4 antagonist is administered prior to the administration of both the STAT3 activator and/or the nitric oxide (NO)level-increasing agent. In yet another embodiment, the CXCR4 antagonist is administered after both the STAT3 activator and the nitric oxide level increasing agent. In still yet another alternative embodiment, the CXCR4 antagonist may be administered in-between the STAT3 activator and the nitric oxide level-increasing agent.

The subject patient may suffer from a peripheral nervous system deficit, such as a congenital nerve deficit or a nerve deficit due to trauma or an iatrogenic event. Alternatively, the peripheral nerve deficit may be due to infection or to an autoimmune disease. Furthermore, the subject patient may suffer from a central nervous system deficit, such as a situation in which the nerve deficit is in the brain or spinal cord, e.g., where the peripheral nerve deficit may consist of a nerve deficit in a cranial nerve or a spinal nerve. The spinal nerve deficit may be congenital, genetic, or due to trauma or an iatrogenic event. The spinal nerve deficit may be due to infection or to autoimmune disease. The spinal nerve deficit may be a cervical deficit, a lumbosacral deficit, or a thoracic deficit. The subject may a non-human animal, such as a bird, a reptile or a mammal, for example, a dog, cat, horse, or cow. The most preferred embodiment will be administration of the method of the disclosure to a subject patient that may be a human.

The method of the present disclosure may further comprise treating said subject patient with physical therapy or other nerve deficit therapy prior to, at the time of, or post-administration of the method of the disclosure, according to methods and regimens well-known to those of ordinary skill in surgery, physical medicine, physical therapy, or occupational therapy. The administration of the method and compositions used in alternative embodiments of the present disclosure may result in improved sensory function in said subject patient, for example such as nociceptive function and/or mechanoceptive function. The administration may result in improved motor control in said subject, such as fine motor control, gross motor control or autonomic nerve control.

It is contemplated that any method or composition described herein can be implemented in coordination with respect to any other method or composition described herein, in any order of procedure, or in any combination of two, three, or more compositions used in the method of the disclosure.

The use of the word “a” or “an” when used in conjunction with the term “comprising” in the claims and/or the specification may mean “one,” but it will also be interpreted herein as also consistent with the meaning of “one or more,” “at least one,” and “one or more than one.” It is contemplated that any embodiment discussed in this specification can be implemented with respect to any method or composition of the disclosure, and vice versa.

Furthermore, compositions and kits of the disclosure can be used to achieve methods of the disclosure. Throughout this application, the term “about” is used to indicate that a value includes the inherent variation of error for the device, the method being employed to determine the value, or the variation that exists among the study subjects.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a device, or a method that “comprises,” “has,” “contains,” or “includes” one or more elements possesses those one or more elements but is not limited to possessing only those one or more elements or steps. Likewise, an element of a device or method that “comprises,” “has,” “contains,” or “includes” one or more features possesses those one or more features but is not limited to possessing only those one or more features.

Although the following detailed description contains many specifics for the purposes of illustration, anyone of ordinary skill in the art will appreciate that many variations and alterations to the following details are within the scope of the disclosure. Accordingly, the following embodiments of the disclosure are set forth without any loss or diminution of generality to, and without imposing limitations upon, the claimed disclosure.

As used herein, the words “exemplary” or “illustrative” mean “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art on how to make or use the embodiments of the disclosure and are not intended to limit the scope of the disclosure, which is defined solely by the claims.

Furthermore, in this detailed description, a person skilled in the art should note that quantitative qualifying terms such as “generally,” “substantially,” “mostly,” and other terms are used, in general, to mean that the referred-to object, characteristic, or quality constitutes a majority of the subject of the reference. The meaning of any of these terms is dependent upon the context within which it is used, and the meaning may be expressly modified. Wherever the word “can” is used, it should be taken to be interchangeable with the word “may”, both being used in the generic possibility sense.

The terms “peripheral nervous system” and “central nervous system” will have their ordinary medical dictionary definitions.

The term “neuronal population” means one or more neuronal cells that may occupy one or more distinct spatial locations in some portion of the anatomy; or may be collectively recognized by those of skill in the neurology sciences and medicine as being of common anatomical, functional, ontogenetic, chemical, or electrically addressable properties.

The terms “nitric oxide enhancement”, “nitric oxide increasing”, “nitric oxide raising”, “nitric oxide production inducer”, and “nitric oxide generator” are used interchangeably and have the same meaning as to their biochemical mechanism of action.

### DETAILED DESCRIPTION

Nerve deficits and injuries constitute a major source of misery, discomfort, immobility, occupational deficit, and attack on the quality of life for the patient, and present a major challenge for health care providers, and, of course, represent a tremendous financial strain on family wealth, insurance company resources, and government agency taxpayer-sourced funds. One significant example is spinal cord injury (SCI), which commonly results in permanent paralysis and sensory impairments due to poor spontaneous nerve regeneration in the central nervous system. This is exacerbated when the injury results in tissue loss due to the trauma. This is normally followed by cell death at the injury epicenter, which results in the formation of a fluid filled local cyst that prevents axonal regeneration. Another significant example is that of nerve injury in the upper and lower extremities, which also results in permanent paralysis and sensory impairments due to poor spontaneous nerve regeneration in the peripheral nervous system. Even when a nerve is cut and directly repaired, the recovery is usually sub-optimal, and the recovery thus represents only a fraction of pre-injury functionality. This is exacerbated when the injury results in tissue loss caused by the trauma. The nerve then has no potential for spontaneous regeneration across this nerve tissue defect, or nerve gap, and with no potential for spontaneous regeneration to bridge the gap, the patient is then left at their maximal degree of harm done by the injury.

Multiple strategies have been proposed in the prior art to repair nerve gap injuries. These include mechanical materials and devices, including the use of scaffolds, lattices, spongiform matrices, or hollow conduits or bundles of multiple hollow conduits made of degradable or nondegradable materials such as PLGA, PGE, multiluminal agarose hydrogels, or silicone. Another strategy, alone or in combination with another technology are the use of applied exogenous growth factors, matrix molecules, or carrier cells (e.g., recombinant cells) that may deliver warhead molecules that may entice nerve regeneration. Surgical implantation of scaffolds or hollow conduits alone permits regeneration across nerve gaps of 1-3 cm, but use of exogenous cells, or exogenous molecules plus scaffolds, are now known to be required for regeneration across larger nerve gaps, known as critical gaps. In the presently disclosed and claimed disclosure, as well as in all its alternative embodiments, the inventors have identified a new therapeutic approach to address such previously untreatable nerve deficits. Using immunomodulatory compounds, and more preferably using a cocktail of different classes of immunomodulatory compounds that may include one or more species of the genus of CXCR4 antagonists, the genus of STAT3 activators and/or the genus of NO-increasing agents, the inventors have been able to achieve nerve regeneration across an empty critical gap greater than 3 cm in length.

The existing paradigms for treating nerve gaps of up to 3 cm in length using scaffolds, hollow conduits, or other mechanical structural components as described hereinabove, typically fail to bridge these critical gaps, which has been the highest barrier or hurdle to overcome in the field of nerve regeneration therapy and has been previously impossible to overcome without the use of exogenous cells, or exogenous molecules plus scaffolds. The introduction of exogenous cells always raises a risk of adverse immune reaction to their presence, and so should only be used when believed to be of greater benefit to the patient than the prior art approach of using such cells as a monotherapy. Distances of less than the critical gap can be easily overcome, even in the absence of exogenous cells or molecules. In contrast, the inventors have now shown that the use of single or combinatorial immunomodulatory drug treatments induces whole tissue nerve regeneration across distances greater than the 3 cm critical gap, and importantly, can do so without the addition of scaffolds or exogenous cells. These and other aspects of the disclosure are described in detail below.

Nerve injury, broadly defined, is injury to nervous tissue. There is no single classification system in neurology that can describe all the many variations of nerve injury. However, in 1941, Seddon introduced the classification system, now named for him, of nerve injuries based on three main types of nerve fiber injury and whether there is continuity of the nerve. First, Neuropraxia, which is a type of nerve injury usually secondary to compression pathology; this is the mildest form of peripheral nerve injury with minimal structural damage; it allows for a complete and relatively short recovery period. In a neuropraxic nerve injury, a focal segment is demyelinated at the site of injury, though with no injury or disruption to the neuronal cell's axon or its surroundings. This is usually due to prolonged ischemia from excess pressure or stretching of the nerve with no Wallerian degeneration (a well-orchestrated morphologic and biochemical change that occurs in axons, Schwann cells, and macrophages distal to the site of a nerve injury, resulting in the establishment of a microenvironment supportive of axonal regeneration). Secondly, Axonotmesis, which is an injury that involves the axon and its myelin sheath; although the internal structure of the neuron is preserved, the damage done to the axons does lead to Wallerian degeneration; while this type of nerve injury also results in complete recovery, it takes longer than does a neuropraxic injury. Thirdly, there is Neurotmesis, which can be a disruption of the axon and endoneurium, and in its most severe occurrence, is a complete disruption of the entire nerve trunk, i.e., the nerve trunk is cut, torn, degenerated, or malformed clear through. Usually, however, (peripheral) nerve injury is classified in one of the Sunderland five stages, based on the extent of damage to both the nerve and the surrounding connective tissue, since supporting glial cells may be involved.

Unlike the situation in the central nervous system, neuroregeneration in the peripheral nervous system is possible. The processes that occur in peripheral regeneration can be divided into the following major events: Wallerian degeneration, axon regeneration/growth, and end-organ reinnervation. The events that occur in peripheral regeneration occur with respect to the axis of the nerve injury. The proximal stump refers to the end of the injured neuron that is still attached to the neuron cell body; that is the part that regenerates. The distal stump refers to the end of the injured neuron that is still attached to the end of the axon; it is the part of the neuron that will degenerate but that remains in the area toward which the regenerating axon grows. Neuropraxia, being the least severe form of nerve injury, may achieve complete recovery. In this case, the axon remains intact, but there is myelin damage causing an interruption in conduction of the impulse down the nerve fiber. Most commonly, this involves compression of the nerve or disruption to the blood supply (ischemia). Since the axon remains intact, the methods of the present disclosure are not applicable to this category of nerve injury. In neuropraxia, there is a temporary loss of function which is reversible within hours to months of the injury (the average is 6-9 weeks). Wallerian degeneration does not occur, so recovery does not involve actual regeneration, which is one of the defining features of the present disclosure. In electrodiagnostic testing done with nerve conduction studies, there is a normal compound motor action potential amplitude distal to the lesion at day 10, and this indicates a diagnosis of mild neuropraxia instead of axonotmesis or neurotmesis.

Axonotmesis is a more severe nerve injury with disruption of the neuronal axon, but with maintenance of the epineurium. This type of nerve damage may cause loss of the motor, sensory, and autonomic functions. This is mainly seen in crush injury. If the force creating the nerve damage is removed in a timely fashion and the surrounding tissue is preserved, the axon may regenerate, leading to recovery. Electrically, the nerve shows rapid and complete degeneration, with loss of voluntary motor units. Regeneration of the motor end plates will occur, so long as the endoneural tubules are intact. Axonotmesis involves the interruption of the axon and its covering of myelin but there is preservation of the connective tissue framework of the nerve (the encapsulating tissue, the epineurium and perineurium, are preserved). Because axonal continuity is lost however, Wallerian degeneration occurs. Electromyography (EMG) performed 2 to 4 weeks later shows fibrillations and denervation potentials in musculature distal to the injury site. Loss in both motor and sensory functions is more complete with axonotmesis than with neurapraxia, and recovery occurs only through regeneration of the axons, a process requiring time.

Axonotmesis is usually the result of a more severe crush or contusion than neuropraxia, but can also occur when the nerve is stretched (without damage to the epineurium). There is usually an element of retrograde proximal degeneration of the axon, and for regeneration to occur, this loss must first be overcome. The regenerating fibers must, through their growth, cross the injury site and regeneration through the proximal or retrograde area of degeneration may require several weeks. Then, the neurite tips progress down to the distal site, such as with the wrist or hand. Proximal lesions may grow distally as fast as 2 to 3 mm per day and distal lesions as slowly as 1.5 mm per day. Regeneration occurs over weeks to years.

Neurotmesis is the most severe lesion with no potential of full recovery. It occurs on severe contusion, stretch, laceration, or local anesthetic toxicity. The axon and encapsulating connective tissue lose their continuity. The last (extreme) degree of neurotmesis is transection. Denervation changes recorded by EMG are the same as those seen with axonotmetic injury. There is a complete loss of motor, sensory and autonomic function. If the nerve has been completely severed, axonal regeneration causes a neuroma to form in the proximal stump.

The following examples are exemplary of nerve defects that may be addressed, in whole or in part, by the practice of a preferred embodiment of the disclosure and claimed herein.

Spinal Cord Nerve Defects and Deficits. A spinal cord injury (SCI) or defect is an injury to the spinal cord resulting in a disruption, either temporary or permanent, in the cord's normal motor, sensory, or autonomic function. Common causes of neuronal damage include trauma (car accident, gunshot, falls, sports injuries, etc.), disease (e.g., transverse myelitis, acute flacid myelitis brachial neuritis, polio, spina bifida, Friedreich's ataxia, etc.), congenital defects, and deformities of genetic origin. The spinal cord does not have to be completely severed in order for a loss of function to occur. Depending on where the spinal cord and nerve roots are damaged, symptoms can vary widely, from pain to paralysis to incontinence. Spinal cord injuries are described at various levels of “incomplete,” which can vary from having no effect on the patient to a “complete” injury which means a total loss of function. Treatment of spinal cord injuries starts with restraining the spine and controlling inflammation to prevent further damage. The actual treatment can vary widely depending on the location and extent of the injury. In many cases, spinal cord injuries require substantial physical therapy and rehabilitation, especially if the patient's injury interferes with activities of daily life, and adjunct treatment comprising the application of physical therapy. Research into treatments for spinal cord injuries includes nerve regeneration through the use of nerve growth factors, controlled hypothermia and stem cells, though many treatments have not been studied thoroughly and very little new research has been reported.

Brain Injury and Cranial Nerve Deficits. Brain damage or brain injury (B) is the destruction or degeneration of brain cells, including neuronal cells. Brain injuries may occur due to a wide range of internal or external factors. A common category with the greatest number of injuries therein is traumatic brain injury (TBI) following physical trauma or head injury applied from an outside source, and the term acquired brain injury (ABI) is now used in diagnostic, treatment, research, and product development circles to differentiate brain injuries occurring after birth from injury due to a congenital disorder or malady.

In general, brain damage refers to significant, undiscriminating trauma-induced damage, while neurotoxicity typically refers to selective, chemically induced neuronal damage. Brain injuries occur due to a very wide range of conditions, illnesses, injuries, and as a result of iatrogenesis (adverse effects of related or unrelated medical treatment). Possible causes of widespread brain damage include birth hypoxia, prolonged hypoxia (shortage of oxygen), poisoning by teratogens including alcohol, infection, and neurological illness. Chemotherapy can cause brain damage to the neural stem cells and oligodendrocyte cells that produce myelin. Common causes of focal or localized brain damage are physical trauma (traumatic brain injury, stroke, aneurysm, surgery, or other neurological disorder), and poisoning from heavy metals including the commonly occurring toxins mercury, arsenic, antimony, beryllium, cadmium, hexavalent chromium, and lead, and those compounds containing these elements.

Cranial nerve disease is an impaired functioning of one of the twelve cranial nerves. It is possible for a disorder of more than one cranial nerve to occur at the same time, if a trauma occurs at a location where many cranial nerves run together, such as the jugular fossa. A brainstem lesion could also cause impaired functioning of multiple cranial nerves, but this condition would likely also be accompanied by distal motor impairment.

The facial nerve is the seventh of 12 cranial nerves. This cranial nerve controls the muscles in the face. Facial nerve palsy is more abundant in older adults than in children and is believed to affect 15-40 out of 100,000 people per year. This disease comes in many forms which include congenital, infectious, traumatic, neoplastic, or idiopathic. The most common cause of such cranial nerve damage is Bell's palsy (idiopathic facial palsy) which is a paralysis of the facial nerve. Although Bell's palsy is more prominent in adults, it seems to be found in those younger than 20 or older than 60 years of age. Bell's palsy is thought to occur by herpes virus infection, which may cause demyelination, and has been found in patients with facial nerve palsy. Symptoms include flattening of the forehead, sagging of the eyebrow, and difficulty closing the eye and the mouth on the side of the face that is affected. The inability to close the mouth causes problems in feeding and speech. It also causes lack of taste, lacrimation, and sialorrhea.

Peripheral Nerve Deficits. Peripheral nerve damage is categorized in the Seddon classification based on the extent of damage to both the nerve and the surrounding connective tissue since the nervous system is characterized by the dependence of neurons on their supporting glia. Unlike in the central nervous system, regeneration in the peripheral nervous system is possible (in the central nervous system, re-routing of signaling over central nervous system neural cells or populations is a target of current research). The processes that occur in peripheral nervous system regeneration can be divided into the following major events: Wallerian degeneration; axon regeneration/growth; and then end-organ reinnervation. The events that occur in peripheral regeneration occur with respect to the axis of the nerve injury. A discontinuity in a neuronal cell creates two stumps. The proximal stump refers to the end of the injured neuron that is still attached to the neuron cell body; it is the part of the neuronal cell that regenerates. The distal stump refers to the end of the injured neuron that is still attached to the end of the axon; it is the part that will degenerate but is the direction and area that the regenerating axon may grow towards. The lowest degree of nerve injury, in which the nerve remains intact though its signaling ability is damaged, is called neurapraxia. The next higher second degree of nerve injury, in which the axon is damaged, but the surrounding connecting tissue remains intact, is called axonotmesis. The highest degree of nerve injury, in which both the axon and connective tissue are damaged, is called neurotmesis.

Immunomodulatory Drugs. As discussed hereinabove the inventors of the present disclosure have determined that CXCR4 antagonists, STAT3 activators, and NO-increasing agents are three pharmacologically distinct genuses of chemical compounds, that all have independent immunomodulatory pharmacologic function to promote nerve growth and regeneration, that is to say, that any one genus has such activity regardless of the absence of one or both of the other two. Therefore, the preferred alternative embodiments of the present disclosure contemplate the use of agents from these three genuses, either individually, or in combination with one or both of the remaining two genuses for treating a wide variety of nerve deficits.

CXCR4 Antagonists. C—X—C chemokine receptor type 4 (CXCR-4), also known as fusin or CD184 (cluster of differentiation 184) is a protein that in humans is encoded by the (XCR4 gene. This protein is a CXC chemokine receptor. CXCR-4 is an alpha-chemokine receptor specific for stromal-derived-factor-1 (SDF-1 also called CXCL12), a molecule endowed with potent chemotactic activity for lymphocytes. CXCR4 is one of several chemokine receptors that HIV can use to infect CD4+ T cells. HIV isolates which use CXCR4 are traditionally known as T-cell tropic isolates. Typically, these viruses are found late in infection. It is unclear as to whether the emergence of CXCR4-using HIV is a consequence or a cause of immunodeficiency.

CXCR4 is upregulated during the implantation window in natural and hormone replacement therapy cycles in the endometrium, producing, in presence of a human blastocyst, a surface polarization of the CXCR4 receptors, suggesting that this receptor is implicated in the adhesion phase of human implantation.

CXCR4's ligand, SDF-1, is known to be important in hematopoietic stem cell homing to the bone marrow and in hematopoietic stem cell quiescence. It has been also shown that CXCR4 signaling regulates the expression of CD20 on B cells. Until recently, SDF-1 and CXCR4 were believed to be a relatively monogamous ligand-receptor pair (other chemokines are promiscuous, tending to use several different chemokine receptors). Recent evidence demonstrates ubiquitin is also a natural ligand of CXCR4. Ubiquitin is a small (76-amino acid) protein highly conserved among eukaryotic cells. It is best known for its intracellular role in targeting ubiquitylated proteins for degradation via the ubiquitin proteasome system. Evidence in numerous animal models suggests ubiquitin is an anti-inflammatory immune modulator and endogenous opponent of proinflammatory damage associated molecular pattern molecules. It is speculated this interaction may be through CXCR4 mediated signaling pathways. Macrophage migration inhibitory factor (MIF) is an additional ligand of CXCR4.

CXCR4 is present in newly generated neurons during embryogenesis and adult life, where it plays a role in neuronal guidance. The levels of the receptor decrease as neurons mature. CXCR4 mutant mice have aberrant neuronal distribution. This has been implicated in disorders such as epilepsy.

Drugs that block the CXCR4 receptor appear to be capable of “mobilizing” hematopoietic stem cells into the bloodstream as peripheral blood stem cells. Peripheral blood stem cell mobilization is very important in hematopoietic stem cell transplantation (as a recent alternative to transplantation of surgically harvested bone marrow) and is currently performed using drugs such as G-CSF. G-CSF is a growth factor for neutrophils (a common type of white blood cells) and may act by increasing the activity of neutrophil-derived proteases such as neutrophil elastase in the bone marrow leading to proteolytic degradation of SDF-1. Plerixafor (AMD3100) is a drug that has been approved by the United States Food and Drug Administration for routine clinical use. AMD3100 directly blocks the CXCR4 receptor. It is a very efficient inducer of hematopoietic stem cell mobilization in animal and human studies. In a small human clinical trial to evaluate the safety and efficacy of fucoidan ingestion (brown seaweed extract), 3 g daily of 75% w/w oral fucoidan for 12 days increased the proportion of CD34+CXCR4+ from 45 to 90% and the serum SDF-1 levels, which could be useful in CD34+ cells homing & mobilization via SDF-1/CXCR4 axis.

WHIM-like mutations in CXCR4 were recently identified in patients with Waldenstrom's macroglobulinemia, a B-cell malignancy. The presence of CXCR4 WHIM mutations has been associated with clinical resistance to ibrutinib in patients with Waldenstrom's Macroglobulinemia.

While CXCR4's expression is low or absent in many healthy tissues, it was demonstrated to be expressed in over 23 types of cancer, including breast cancer, ovarian cancer, melanoma, and prostate cancer. Expression of this receptor in cancer cells has been linked to metastasis to tissues containing a high concentration of CXCL12, such as lung, liver, and bone marrow. However, in breast cancer where SDF1/CXCL12 is also expressed by the cancer cells themselves along with CXCR4, CXCL12 expression is positively correlated with disease-free (metastasis-free) survival. CXCL12 over-expressing cancers might not sense the CXCL12 gradient released from the metastasis target tissues since the receptor, CXCR4, is saturated with the ligand produced in an autocrine manner. Another explanation of this observation is provided by a study that shows the ability of CXCL12 (and CCL2)-producing tumors to entrain neutrophils that inhibit seeding of tumor cells in the lung. Chronic exposure to THC has been shown to increase T lymphocyte CXCR4 expression on both CD4+ and CDS+ T lymphocytes in rhesus macaques. It has been shown that BCR signaling inhibitors also affect CXCR4 pathway and thus CD20 expression. CXCR4 has been shown to interact with USP14.

A CXCR4 antagonist is a substance which blocks the CXCR4 receptor and prevents its activation. Blocking the receptor stops the receptor's ligand, CXCL12, from binding, which prevents downstream effects. CXCR4 antagonists are especially important for hindering cancer progression because of one of the downstream effects that is initiated by CXCR4. CXCR4 receptor activation is a cause of cell movement, which helps the spread of cancer, otherwise known as metastasis. The CXCR4 receptor has been targeted by antagonistic substances since being identified as a co-receptor in HIV and assisting the development of cancer. Macrocyclic ligands have been utilized as CXCR4 antagonists. Plerixafor is an example of a CXCR4 antagonist, and has approvals (e.g., U.S. FDA 2008) for clinical use (to mobilize hematopoietic stem cells). BL-8040 is a CXCR4 antagonist that has undergone clinical trials (e.g., in various leukemias), with one planned for pancreatic cancer (in combination with pembrolizumab). Previously called BKT140, it is a synthetic cyclic 14-residue peptide with an aromatic ring. In a 2018 mouse tumor model study, BL-8040 treatment enhanced anti-tumor immune response potentially by increasing the cos+ T-cells in the tumor microenvironment. WZ 811, an agent with a different molecular structure from Plerixafor, has also been used. It is important to understand that in the practice of the present disclosure in the treatment of a patient that has suffered partial or total discontinuity of a nerve, the inventors have found that administration of a CXCR4 antagonist results in stimulating neural cell growth, which is contrary to the teaching of the prior art that CXCR4 antagonists are useful in the treatment of cancer by inhibiting the growth of cancer cells.

Stat3 Activators. Signal transducer and activator of transcription 3 (STAT3) is a transcription factor which in humans is encoded by the STAT3 gene. It is a member of the STAT protein family. STAT3 is a member of the STAT protein family. In response to cytokines and growth factors, STAT3 is phosphorylated by receptor-associated Janus kinases (JAK), form homo- or heterodimers, and translocate to the cell nucleus where they act as transcription activators. Specifically, STAT3 becomes activated after phosphorylation of tyrosine 705 in response to such ligands as interferons, epidermal growth factor (EGF), Interleukin (IL)-5 and IL-6. Additionally, activation of STAT3 may occur via phosphorylation of serine 727 by Mitogen-activated protein kinases (MAPK) and through c-src non-receptor tyrosine kinase. STAT3 mediates the expression of a variety of genes in response to cell stimuli, and thus plays a key role in many cellular processes such as cell growth and apoptosis.

STAT3-deficient mouse embryos cannot develop beyond embryonic day 7, when gastrulation begins. It appears that at these early stages of development, STAT3 activation is required for self-renewal of embryonic stem cells (ESCs). Indeed, LIF, which is supplied to murine ESC cultures to maintain their undifferentiated state, can be omitted if STAT3 is activated through some other means. STAT3 is essential for the differentiation of the TH17 helper T cells, which have been implicated in a variety of autoimmune diseases. During viral infection, mice lacking STAT3 in T-cells display impairment in the ability to generate T-follicular helper (Tfh) cells and fail to maintain antibody-based immunity.

Loss-of-function mutations in the STAT3 gene result in Hyperimmunoglobulin E syndrome, associated with recurrent infections, as well as disordered bone and tooth development. Gain-of-function mutations in the STAT3 gene have been reported to cause multi-organ early onset auto-immune diseases; such as thyroid disease, diabetes, intestinal inflammation, and low blood counts, while constitutive STAT3 activation is associated with various human cancers and commonly suggests poor prognosis. It has anti-apoptotic as well as proliferative effects.

STAT3 can promote oncogenesis by being constitutively active through various pathways as mentioned elsewhere. A tumor suppressor role of STAT3 has also been reported. In the report on human glioblastoma tumor, or brain cancer, STAT3 was shown to have an oncogenic or a tumor suppressor role, depending upon the mutational background of the tumor. A direct connection between the PTEN-Akt-FOXO axis (suppressive) and the leukemia inhibitory factor receptor beta (LIFRbeta)-STAT3 signaling pathway (oncogenic) was shown. Increased activity of STAT3 in cancer cells leads to changes in the function of protein complexes that control expression of inflammatory genes, with resultant profound change in the secretome and the cell phenotypes, their activity in the tumor, and their capacity for metastasis. Niclosamide seems to inhibit the STAT3 signalling pathway. STAT3 has been shown to interact with AR, ELP2, EP300, EGFR, HIFIA, JAK1, JUN, KHDRBSI, mTOR, MYODI, NDUFA13, NFKBI, NR3CI, NCOAI, PML, RACI25, RELA, RET, RPA2, STATI, Stathmin, Src, TRIPIO and KPNA4.

STAT3 activators include colivelin, and neuroprotective peptide, ruxolitinib phosphate, a JAK1/JAK2 inhibitor, or IL-6.

NO Promoting Agents. Nitric oxide (nitrogen oxide or nitrogen monoxide) in its free state is a colorless gas with the formula NO. It is one of the principal oxides of nitrogen. Nitric oxide is a free radical, i.e., it has an unpaired electron, which is sometimes denoted by a dot in its chemical formula, i.e., ·NO. Importantly, since ·NO is a free radical, it is a notorious inflammatory agent. Nitric oxide is a signaling molecule that plays a key role in the pathogenesis of inflammation. Whereas it has an anti-inflammatory effect under normal physiological conditions, on the other hand, NO is a pro-inflammatory mediator that induces inflammation due to overproduction in abnormal situations, including injury to neuronal cells, along with the collateral injuries attendant thereto. The prior art teaches that NO is synthesized and released into endothelial cells by the help of NOSs that convert arginine into citrulline-producing NO in the process. NO is believed to induce vasodilatation in the cardiovascular system and furthermore, it is involved in immune responses by cytokine-activated macrophages, which release NO in high concentrations. In addition, NO is a potent neurotransmitter at the neuron synapses and contributes to the regulation of apoptosis. NO is involved in the pathogenesis of inflammatory disorders of the joint, gut, and lungs. Therefore, NO inhibitors represent important therapeutic advances in the management of inflammatory diseases. Selective NO biosynthesis inhibitors are proven to be useful in the treatment of NO-induced inflammation. J. N. Sharma et al., Role of Nitric Oxide in Inflammatory Diseases, Inflammopharmacology, 15(6), 252-9, 2007. In view of the teaching of the prior art, it is important to understand that in the practice of the present disclosure in the treatment of a patient that has suffered discontinuity of a nerve, the inventors have found that administration of a NO promoter results in stimulating neural cell growth, which is contrary to the teaching of the prior art that NO is prominent in causing inflammation under conditions of trauma, notably neural damage trauma.

Nitric oxide is also a heteronuclear diatomic molecule, a historic class that drew the attention of researchers, and which spawned early modem theories of chemical bonding. An important intermediate in chemical industry, nitric oxide forms in combustion systems and can be generated by lightning in thunderstorms. In mammals, including humans, nitric oxide is a signaling molecule in many physiological and pathological processes. Nitric oxide should not be confused with nitrous oxide (N₂O), an anesthetic, nor with nitrogen dioxide (NO₂), a brown toxic gas and a major air pollutant. NO promoting donor agents, which have their anti-inflammatory therapeutic place in normal physiologic conditions, include (+/−)-S-Nitroso-N-acetylpenicillamine, Molsidomine, 3-Morpholinosydnonimine, Hydroxyguanidine sulfate, Tetrahydrobiopterin (THB) dihydrochloride, S-Nitrosoglutathione (GSNO), Streptozotocin (U-9889), Nicorandil, Dephostatin, DETA NONOate, NOC-12, NOC-18, NOC-5, NOC-7, MAHMA NONOate, PAPA NONOate, Sulfo-NONOate disodium salt, Angeliprimes salt, Diethylamine NONOate, NOR-1, NOR-2, NOR-3, NOR-4, Spermine NONOate, beta-Gal NONOate, BNN3, GEA 3162, GEA 5024, Sodium nitroprusside dihydrate, 10-Nitrooleate, BEC, NO-Indomethacin, Pilotyprimes Acid, SE 175, V-PYRRO/NO, Vinyl-L-NIO Hydrochloride, AMI-1, sodium salt, DAF-FM DA (cell permeable), GEA 5583, N-Acetyl-D,L-penicillamine disulfide, SIN-1A/gammaCD Complex, 4-Phenyl-3-furoxancarbonitrile, JS-K, Lansoprazole Sulfone N-Oxide, NO-Aspirin 1, Glyco-SNAP-2, N,N-Dicarboxymethyl-N,N-dinitroso-p-phenylenediamine (Disodium Salt), (2S)-(+)-Amino-6-iodoacetamidohexanoic acid, 4AF DA, BEC ammonium salt, DAF-2 DA (cell permeable), DAN-1 EE hydrochloride, DD1, DD2, Diethylamine NONOate/AM, Fructose-SNAP-1, Glyco-SNAP-1, Guanylyl Cyclase, Hydroxyguanidine hemisulfate, N-Cyclopropyl-N′-hydroxyguanidine hydrochloride, NOR-5, PROLI, NONOate, S-Nitrosocaptopril, 4-(p-methoxyphenyl)-1,3,2-Oxathiazolylium-5-olate, 4-chloro-4-phenyl-1,3,2-Oxathiozolylium-5-olate, 4-phenyl-1,3,2-Oxathiazolylium-5-olate, 4-trifluoro-4-phenyl-1,3,2-Oxathiazolylium-5-olate, Tricarbony Idichlororuthenium(II) dimer, DL-alpha-Difluoromethylomithine hydrochloride, Geranylgeranylacetone, N-Nitrosodiethylamine, L-NMMA (citrate), and 3-(Methy Initrosamino)propionitrile. SIN-1 chloride, L-Arginine, and SNAP. Other drug classes can also serve to increase local concentrations of nitric oxide, including nitroglycerin, isosorbide dinitrate, isosorbide mononitrate, or PDE5 inhibitors (e.g., sildenafil, tadalafil, and the like). Another NO-promoting agent is L-arginine, which is a substrate for NO synthase.

Pharmaceutical Formulations and Methods of Administration. Where clinical applications are contemplated, it will be necessary to prepare pharmacologically active compositions and inert adjuvants in a pharmaceutical formulation that is appropriate for the intended application. Generally, this will entail preparing compositions that are essentially free of pyrogens, as well as other impurities that could be harmful to exposed tissues and cells in humans or animals. The active compositions of the present disclosure may include classic pharmaceutical preparations. One will generally desire to employ appropriate salts and buffers to render agents stable and allow for uptake by target cells. Aqueous compositions of the present disclosure comprise an effective amount of the monotherapy agent or fixed dose combination of agents to cells, dissolved or dispersed in a pharmaceutically acceptable carrier or aqueous medium. Such compositions are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for delivering pharmaceutically active substances is routine and well known in the art, unlike the process of selecting an active ingredient and finding its optimum concentration in an inocula, a process that is entirely unpredictable and has no reasonable expectation of success without experimental observation and verification. One of ordinary skill in the art may have an understanding of those classes of molecules that may more quickly lead to a discovery or a disclosure, and of their structure-activity-relationships, but again, no one knows for certain whether administration to a patient will be safe and efficacious until an adequately well-designed, reviewed, conducted, observed, and reported trial has been completed. Except insofar as any conventional media or agent is incompatible with the present disclosure, its use in therapeutic compositions is contemplated. Supplementary active ingredients also can be incorporated into the compositions used in the methods of the present disclosure.

Administration of these compositions according to the present disclosure will be via an appropriate route but are particularly drawn to administration at a local or regional proximity to a nerve deficit. Administration may be by injection, infusion, implantation, insertion, or application during a surgical procedure. Such compositions would normally be administered as pharmaceutically acceptable compositions. When the route of administration is topical, the form may be a solution, lotion, emulsion, cream, ointment, or salve. An effective amount of the therapeutic agent is determined based on the intended therapeutic goal, i.e., initiating nerve growth, improving nerve growth, sustaining nerve growth, all to the goal of reducing a nerve deficit, and thus bridging a “critical gap.” The term “unit dose” refers to physically discrete units suitable for use in a subject, each unit containing a predetermined quantity of the therapeutic composition calculated to produce the desired responses, discussed above, in association with its administration, i.e., the appropriate route and treatment regimen. The quantity to be administered, both according to number of procedures, treatments and unit dose, depends on the subject to be treated, the state of the subject and the protection desired. Precise amounts of the therapeutic composition also depend on the judgment of the practitioner and are peculiar to each individual patient.

As used herein, the term in vitro preparation refers to manipulations performed on materials outside of the living patient. The term ex vivo administration refers to materials that have been manipulated in vitro and are subsequently administered to a living animal. The term in vivo administration includes all manipulations performed within a patient. In certain aspects of the present disclosure, the compositions may be prepared in vitro or administered either ex vivo or in vivo. In the most preferred embodiment of the disclosure, the special case of surgical intervention, the present disclosure may be used preoperatively, during surgery, or post-operatively. The administration may be continued post-surgery, for example, by leaving a catheter implanted at the site of the surgery. Such post-surgical treatment administered periodically also is envisioned. Generally, the dose of the therapeutic composition via continuous perfusion will be equivalent to that given by single or multiple injections, adjusted over a period of time during which the perfusion occurs. Treatment regimens may vary as well, and often depend on deficit type, deficit location, and health and age of the patient. Obviously, certain types of deficits will require more aggressive treatment, while at the same time, certain patients cannot tolerate more taxing protocols. The clinician will be best suited to make such decisions based on the known efficacy and toxicity (if any) of the therapeutic formulations. Solutions of the active compounds in the form of free base or pharmacologically acceptable salts can be prepared in sterile water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a non-toxic preservative to prevent the growth of microorganisms. The therapeutic compositions of the present disclosure are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are dimethyl sulfoxide, propylene glycol, polyethylene glycol, vegetable oil and injectable organic esters such as ethyloleate. Aqueous carriers include water, alcoholic/aqueous solutions, saline solutions, parenteral vehicles such as sodium chloride or Ringer's dextrose. Intravenous vehicles include fluid and nutrient replenishers. Preservatives include antimicrobial agents, antioxidants, chelating agents and inert gases. The pH and exact concentration of the various components the pharmaceutical composition can be adjusted according to well-known parameters.

Combination Therapy. The inventors have determined that combinations of the aforementioned agents, whether a pairwise combination, or a triple compound combination, are particularly efficacious in addressing nerve deficits and promoting nerve growth and regeneration. These compositions would be provided in a combined amount effective to accomplish any or all of the foregoing goals. This process may involve providing agent(s) or factor(s) to a cell, tissue or subject at the same time. This may be achieved by treating the cell, tissue, or subject with one or more compositions or pharmacological formulations that include two or three agents, or by treating the cell, tissue, or subject with one, two or three distinct compositions or formulations. Alternatively, the various agents may precede or follow the second (and/or third) agent or treatment by intervals ranging from minutes to weeks. In embodiments where the delivery, such that the second (and/or third) agent and the first agent would still be able to exert an advantageously combined effect on the cell, tissue, or subject. In such instances, it is contemplated that one would treat the cell, tissue, or subject with multiple modalities within about 12-24 hr of each other and, more preferably, within about 6-12 hr of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. It also is conceivable that more than one administration of the first and/or the second and/or the third agent will be desired.

Nerve Growth Support Structures and Other Nerve Promoting Agents. In certain embodiments, the inventors contemplate inserting supports into the site of nerve deficits in order to provide a substrate upon and/or through which nerves may regrow. The support structures may be combined with other features, such as bioactive materials that are positioned within or adjacent to the support structure, as well as various biological factors that may stimulate, promote or improve nerve growth.

Conduits. Conduits are walled tubes creating an inner bore or lumen and the lumen space is spatially defined by elongated tubular structures with open ends and a lumen passing therethrough. While the exemplified conduits have a circular cross-section, they may be other shapes as well, such as oval, square, rectangular or hexagonal. The conduits may be rigid to semi-rigid in nature, sustaining a force of 100 kPa to 2.0 GPa. They may be biodegradable, non-biodegradable, or at least not biodegradable for a period of months to years following implantation. The conduits may be formed from poly-lactide acid, polyurethane, silicone, cellulose, collagen, poly-lactide co-glycolic acid, polycaprolactone or processed natural extracellular matrix. The conduits may be from about 0.5 mm to about 6.5 cm or greater in length, from about 1.5 mm to about 4.0 mm in external diameter, and with a lumen of from about 1.5 mm to about 3.0 mm internal diameter. The conduit wall may be about 0.2 mm to about 0.6 mm in thickness. The conduits may further be coated internally with bioactive materials or nerve growth enhancing agents, as discussed below. For example, collagen and other extracellular matrix components are contemplated as materials to coat such conduits.

Polymer Fibers. In another alternative preferred embodiment, the structure will be composed of polymer fibers that act as a regenerative guide for growing/regrowing nerve tissue. The fibers will act as a more traditional scaffold, with nerves growing on top of, or around the guide. Suitable polymers include poly-lactide acid, polyurethane, silicone, cellulose, collagen, poly-lactide co-glycolic acid, and polycaprolactone.

Bioactive Materials. In certain preferred alternative embodiments, the support structure may be surrounded or partially surrounded by bioactive materials; and/or the support structure may contain bioactive luminal fillers if it has hollow regions. This bioactive material is solid, semi-solid or in a gel state which can provide further support for the growth of nerve tissue, as well as to serve as a depot for the delivery of growth enhancing agents (discussed below). Suitable substances for the bioactive material include agar, collagen, laminin, fibronectin, or glycoproteins. Such bioactive material can be of uniform nature or can be formulated to contain a differential concentration of molecules such as collagen, laminin, fibronectin, growth factors, biopolymers, and/or pharmacological agents. The bioactive material may be solid, or may contain microparticles, nanoparticles, microcompartments, or microchannels, again selected and engineered to facilitate growth of newly stimulated nerve tissue through the conduit, and to act as a repository for agents. Where there are microcompartments in the lumen, they may in turn be filled with collagen, polymeric microparticles or nanoparticles or fibers and/or cells such as Schwann cells, fibroblasts, immune cells, neurons, stem cells, induced pluripotential cells (IPCs), other autogenous cells, and/or other exogenous cells. These cells can be genetically modified to enhance nerve regeneration such as by expressing growth factors or surface molecules. Such microcompartments can be used also to provide a controlled environment for cells cultured therein prior to implantation, or for those migrating therein after implantation. This environment can consist of incorporated diverse means for the sustained delivery of growth factors, cytokines, anti-inflammatory, and other growth enhancing molecules. Among the growth enhancing molecules incorporated into the structural support may be blockers for growth inhibitory molecules including those designed to block myelin-associated inhibitors (MAG and EphB3), and/or the chondroitin sulphate proteoglycans (CSPG) versican and neurocan.

Bioactive Materials. In certain alternative preferred embodiments, the bioactive materials may be designed to deliver a growth factor. Alternatively, the structural support itself may be coated with a growth factor. These factors may be neurotrophic (e.g., NGF, BDNF, or NT-3), glial-derived (GDNF), or pleotropic (PTN, VEGF).

Nerve growth factor (NGF) is a small, secreted protein that is important for the growth, maintenance, and survival of certain target neuronal cells. NGF also functions as a signaling molecule. NGF is perhaps the prototypical growth factor, in that it is one of the first to have been described. While “nerve growth factor” refers to a single factor, “nerve growth factors” refers to a family of factors that are also known as neurotrophins. Members of the neurotrophin family that are well recognized for their growth-promoting effect include NGF, Brain-Derived Neurotrophic Factor (BDNF), Neurotrophin-3 (NT-3), and Neurotrophin 4/5 (NT-4/5). BDNF is a protein that is encoded by the BDNF gene. BDNF binds to at least two receptors on the surface of cells that are capable of responding to this growth factor, as well as to TrkB and the LNGFR (low-affinity nerve growth factor receptor, also known as p75). BDNF may also modulate the activity of various neurotransmitter receptors, including the Alpha-7 nicotinic receptor. BDNF has also been shown to interact with the reelin signaling chain.

Neurotrophin-3 is a protein that is encoded by the NTF3 gene. NT-3 exhibits activity on certain neurons of the peripheral and the central nervous system, helps to support the survival and differentiation of existing neurons, and encourages the growth and differentiation of new neurons and synapses. Neurotrophin-4 (NT-4), also known as neurotrophin-5 (NT-5) or NT-4/5, is encoded by the NTF4 gene. NT-4 is a neurotrophic factor that signals predominantly through the TrkB receptor tyrosine kinase.

The GDNF family of ligands (GFL) consists of four neurotrophic factors, namely glial cell line-derived neurotrophic factor (GDNF), neurturin (NRTN), artemin (ARTN), and Persephin (PSPN). GFLs have been shown to play a role in a number of biological processes including cell survival, neurite outgrowth, cell differentiation, and cell migration. In particular, signaling by GDNF promotes the survival of dopaminergic neurons and potently promotes the survival of many types of neurons.

Pleiotrophin (PTN) is also known as heparin-binding brain mitogen (HBBM) or heparin-binding growth factor 8 (HBGF-8), neurite growth-promoting factor 1 (NEGFI), heparin affinity regulatory peptide (HARP), or heparin binding growth associated molecule (HB-GAM), and is encoded by the PTN gene. PTN is an 18-kDa growth factor that has a high affinity for heparin. PTN is structurally related to midkine and retinoic acid induced heparin-binding protein.

Vascular endothelial growth factor (VEGF), originally known as vascular permeability factor (VPF), is a signal protein produced by cells that stimulate vasculogenesis and angiogenesis. It is part of the system that restores the oxygen supply to tissues when blood circulation is inadequate. Serum concentration of VEGF is high in bronchial asthma and diabetes mellitus. VEGF's normal function is to create new blood vessels during embryonic development, new blood vessels after injury, muscle following exercise, and new vessels (collateral circulation) to bypass blocked vessels.

Examples. The following examples are included to demonstrate the composition and implementation of particular alternative preferred embodiments of the disclosure. It should be appreciated by those of ordinary skill in the art that the techniques disclosed in the following examples represent techniques discovered by the inventors to function well in the practice of the disclosure, and thus can be considered to constitute specifically contemplated modes for its practice. However, those of ordinary skill in the art should, in light of the present disclosure, appreciate that many changes can be made in the specific embodiments which are disclosed herein yet still obtain a like or similar result without departing from the spirit and scope of the disclosure.

Example 1. >350 g male Lewis rats were anesthetized with ketamine/dexmedetomidine IP injection. The right hindlimb was clipped and sterilely prepped. A posterior thigh muscle splitting approach was taken to expose the sciatic nerve. The nerve was transected at midthigh. A 1.5 mm inner diameter, 12 mm long silicone tube was loaded with collagen gel (Millipore Sigma, Burlington, MA)+/−drug and incubated at 37° C. for 1 hr to allow the gel to solidify. The tube was then placed as an interpositional conduit between the cut ends of the sciatic nerve and sutured with 7-0 polypropylene (Ethicon, Raritan, NJ), with 2 mm of length overlap between the nerve and tube at each end for a total nerve gap length of 8 mm. Drug concentrations were as follows: IL6 100 ng/ml (Millipore Sigma, Burlington, MA), SIN-1 1 mM (Tocris, Minneapolis, MN), 1× AMD3100 3 uM and 10× AMD3100 30 uM (Tocris, Minneapolis, MN). Following conduit implantation, the hamstring muscle was reapproximated with a surgical staple and the skin was likewise closed with staples. Anesthesia was reversed with atipamezole IP injection. Following recovery, rats were housed singly with water and sterile chow ad libitum for 6 weeks. At 6 weeks, rats were again anesthetized with ketamine/dexmedetomidine IP injection. The distal nerve stumps were harvested, and immersion fixed in 3% glutaraldehyde at 4° C. and postfixed with 1% osmium tetroxide. Serial dehydration was performed following fixation using ethanol, and specimens were embedded in Araldite 502 and cut into semithin sections followed by staining with 1% toluidine blue dye and mounting onto glass slides for imaging. A Leco IA32 Image Analysis System (Leco, St. Joseph, MO) was used for quantification of nerve samples. This setup was used to calculate the total fascicle area of the nerve specimen. To calculate the total axons, 5 randomly selected high-magnification images (1000×) per sample were used. Statistical analysis was performed using Excel (Microsoft, Redmond, WA). These results indicate that each of the drugs used in isolation (IL6, SIN-1, AMD3100) may increase axon count following short gap (<3 cm nerve gap, i.e., 8 mm gap length) nerve reconstruction by an average of 1.5-5× when compared with controls without drug delivery. In individual animals, the increase may be as high as 9× when compared with controls without drug delivery. The inventors' gene array studies have shown that each of the drugs impacts nerve regeneration through a distinct non-redundant set of gene expression changes, indicating that they increase nerve regeneration through different pathways.

| | Distal Nerve Axon | |
|---|---|---|
| | Count, 8 mm | % |
| Active Agent | gap, 6 weeks | Increase |
| IL6, 100 ng/mL | | 190.1973 |
| | 7512 | |
| | 1944 | |
| | 9224 | |
| SIN-1, 1 mM | | 167.0654 |
| | 9672 | |
| | 3571 | |
| | 3948 | |
| 1x AMD3100, 3 μM | | 148.9669 |
| | 6262 | |
| | 6000 | |
| | 3764 | |
| 10x AMD3100, 30 μM | | 490.8187 |
| | 18692 | |
| | 13707 | |
| | 5632 | |
| Control, 3D Collagen Gel | | — |
| | 2270 | |
| | 1722 | |
| | 2445 | |

Example 2. Following the procedure of Example 1, a 36 mm long silicone tube is loaded with collagen gel and incubated and is then placed as an interpositional conduit between the cut ends of the sciatic nerve and is sutured so as to bridge a total nerve gap of 32 mm. Drug concentrations are as follows: IL6 100 ng/ml, SIN-1 1 mM, 1× AMD3100 3 uM and 10× AMD3100 30 uM. Following conduit implantation, the hamstring muscle is reapproximated with a surgical staple and the skin is closed with staples. Anesthesia is reversed with atipamezole IP injection. Following recovery, rats are housed singly with water and sterile chow ad libitum for 6 weeks. At 6 weeks, rats are again anesthetized with ketamine/dexmedetomidine IP injection. The distal nerve stumps are harvested and immersion fixed in 3% glutaraldehyde at 4° C. and are postfixed with 1% osmium tetroxide. Serial dehydration is performed following fixation using ethanol, and specimens are embedded in Araldite 502 and cut into semithin sections followed by staining with 1% toluidine blue dye and mounting onto glass slides for imaging. The Leco IA32 Image Analysis System is used for quantification of nerve samples. This setup is used to calculate the total fascicle area of the nerve specimen by observation of high magnification images.

Example 3. Following the procedure of Example 1, a 48 mm long silicone tube is loaded with collagen gel and incubated and is then placed as an interpositional conduit between the cut ends of the sciatic nerve and is sutured so as to bridge a total nerve gap of 44 mm. Drug concentrations are as follows: IL6 100 ng/ml, SIN-1 1.1 mM, 1× AMD3100 3.3 uM and 10× AMD3100 33 uM. Following conduit implantation, the hamstring muscle is reapproximated with a surgical staple and the skin is closed with staples. Anesthesia is reversed with atipamezole IP injection. Following recovery, rats are housed singly with water and sterile chow ad libitum for 6 weeks. At 6 weeks, rats are again anesthetized with ketamine/dexmedetomidine IP injection. The distal nerve stumps are harvested and immersion fixed in 3% glutaraldehyde at 4° C. and are postfixed with 1% osmium tetroxide. Serial dehydration is performed following fixation using ethanol, and specimens are embedded in Araldite 502 and cut into semithin sections followed by staining with 1% toluidine blue dye and mounting onto glass slides for imaging. The Leco IA32 Image Analysis System is used for quantification of nerve samples. This setup is used to calculate the total fascicle area of the nerve specimen by observation of high magnification images.

Example 4. Following the procedure of Example 1, a 56 mm long silicone tube is loaded with collagen gel and incubated and is then placed as an interpositional conduit between the cut ends of the sciatic nerve and is sutured so as to bridge a total nerve gap of 52 mm. Drug concentrations are as follows: IL6 100 ng/ml, SIN-1 1.1 mM, 1× AMD3100 3.3 uM and 10× AMD3100 33 uM. Following conduit implantation, the hamstring muscle is reapproximated with a surgical staple and the skin is closed with staples. Anesthesia is reversed with atipamezole IP injection. Following recovery, rats are housed singly with water and sterile chow ad libitum for 6 weeks. At 6 weeks, rats are again anesthetized with ketamine/dexmedetomidine IP injection. The distal nerve stumps are harvested and immersion fixed in 3% glutaraldehyde at 4° C. and are postfixed with 1% osmium tetroxide. Serial dehydration is performed following fixation using ethanol, and specimens are embedded in Araldite 502 and cut into semithin sections followed by staining with 1% toluidine blue dye and mounting onto glass slides for imaging. The Leco IA32 Image Analysis System is used for quantification of nerve samples. This setup is used to calculate the total fascicle area of the nerve specimen by observation of high magnification images.

Example 5. Following the procedure of Example 1, a 64 mm long silicone tube is loaded with collagen gel and incubated and is then placed as an interpositional conduit between the cut ends of the sciatic nerve and is sutured so as to bridge a total nerve gap of 60 mm. Drugs and their concentrations are as follows: IL3 100 ng/ml, SIN-1 1.1 mM, and bortezmib, mM. Following conduit implantation, the hamstring muscle is reapproximated with a surgical staple and the skin is closed with staples. Anesthesia is reversed with atipamezole IP injection. Following recovery, rats are housed singly with water and sterile chow ad libitum for 6 weeks. At 6 weeks, rats are again anesthetized with ketamine/dexmedetomidine IP injection. The distal nerve stumps are harvested and immersion fixed in 3% glutaraldehyde at 4° C. and are postfixed with 1% osmium tetroxide. Serial dehydration is performed following fixation using ethanol, and specimens are embedded in Araldite 502 and cut into semithin sections followed by staining with 1% toluidine blue dye and mounting onto glass slides for imaging. Leco IA32 Image Analysis System is used for quantification of nerve samples. This setup is used to calculate the total fascicle area of the nerve specimen by observation of high magnification images.

Example 6. Following the procedure of Example 1, a 70 mm long silicone tube is loaded with collagen gel and incubated and is then placed as an interpositional conduit between the cut ends of the sciatic nerve and is sutured so as to bridge a total nerve gap of 66 mm. Drug concentrations are as follows: IL6 100 ng/ml, SIN-1 1.1 mM, 1× AMD3100 3.3 uM, 5× AMD3100 16.5 uM and 10× AMD3100 33 uM. Following conduit implantation, the hamstring muscle is reapproximated with a surgical staple and the skin is closed with staples. Anesthesia is reversed with atipamezole IP injection. Following recovery, rats are housed singly with water and sterile chow ad libitum for 6 weeks. At 6 weeks, rats are again anesthetized with ketamine/dexmedetomidine IP injection. The distal nerve stumps are harvested and immersion fixed in 3% glutaraldehyde at 4° C. and are postfixed with 1% osmium tetroxide. Serial dehydration is performed following fixation using ethanol, and specimens are embedded in Araldite 502 and cut into semithin sections followed by staining with 1% toluidine blue dye and mounting onto glass slides for imaging. The Leco IA32 Image Analysis System is used for quantification of nerve samples. This setup is used to calculate the total fascicle area of the nerve specimen by observation of high magnification images.

Silicone tubing. A tube of silicone is biologically inert and has been widely used in the art for nerve regeneration studies (Lundborg et al., 1982; Williams et al., 1983). Lundborg showed that the rat sciatic nerve never regenerates across an empty silicone tube when presented with a gap longer than 15 mm. The inventors, however, have shown that the rat sciatic nerve can regenerate across at least 30 mm in an empty (empty of nerve tissue) silicone tube when the lumen of the silicon tube has been packed with one or more of the preferred compositions in the practice of the disclosure, and then inserted into the nerve gap to be bridged. Again, a 30 mm gap was selected since it is the longest non-regenerative distance to be bridged when using the methods and compositions of the prior art, and thus represents at a minimum, what was believed to be the most difficult nerve regeneration condition up to the time of the conception of the disclosure. Furthermore, this nerve length of 30 mm is conserved across all species (Strauch et al., 2001) thus supporting the inventors' belief in the limitations of the prior art treatments. Surprisingly, unpredictably, and unexpectedly, the inventors have shown that the rat sciatic nerve can regenerate across a gap of at least 50 mm in an empty silicone tube that has been treated and implanted in accordance with the methods of the methods disclosed herein. Furthermore, the inventors have shown that the methods disclosed herein can also improve the outcomes of the simplest types of nerve injuries, as represented by a sciatic nerve cut and immediate repair.

The concept of using a tube to connect two severed nerve stumps is inherently simple and appealing: nerve stumps are pulled into the ends of a tube- or tube-like structure-which restricts the direction of regenerating axonal processes towards the distal nerve segment while at the same time protecting the regenerating axons from intervening scar tissue, inflammatory process cells, or trauma. It is well-known in the art that the protected microenvironment created by the conduit can contain an enriched milieu of concentrated neurotrophic factors. The present disclosure addresses the need for effective neurotrophic factors to form at least part of the matrix or filler of a neural conduit.

With no convincing clinical data that one commercially available conduit is better than another, the important parameters to keep in mind when considering conduit repair are nerve diameter and gap size. Regardless of the size of the nerve being repaired, the selected conduit diameter should closely fit the stumps. The use of an undersized conduit is not only conceptually poor given the likely resultant constriction of the regenerating nerve, but also technically difficult for the surgeon to squeeze the stump into a tight conduit.

As provided above. AMD3100, the use of which is a preferred embodiment of the present disclosure, is an antagonist with the CXCR4 chemokine receptor (Gerlach, et al., J. Biol. Chem. (2001) 276.14153-14160). This compound is now known to, for example, interfere with the binding of bone marrow stromal cell-derived SDF-1, with CXCR4 on stem cells, which leads to the release of hematopoietic stem cells from bone marrow into the circulation (Broxmeyer, et al., Blood (2001) 98:811a (Abstract)). In a Phase I study at the University of Washington, Seattle, a single dose of 80 μg/kg of AMD3100 resulted in a WBC count of 17,000/μl and a peak 6-fold increase m circulating CD34+ progenitor/stem cells at the 6-hour time point (Liles, et al., Blood (2001) 98:737a (Abstract)). In another recent study mice were injected with rhG-CSF and recombinant rat Stem Cell Factor (rrSCF) in order to mobilize large numbers of hematopoetic bone marrow stem cells into the circulation, after which point a heart attack was induced. The combination of rrSCF and rhG-CSF provided a peak number of circulating stem cells after 5 daily injections. At 27 days post-surgery, there was a 68% Improvement in survival in the treated group versus the controls. At this time the dead tissue was replaced with regenerating myocardium and all functional parameters tested were improved compared with controls (Orlic, et al., PNAS (2001) 98:10344-10349). These studies are shown to demonstrate that when the CXCR4 chemokine receptor is antagonized, one of the five categories of stem cells may be stimulated to be released, but it should be noted that the present disclosure targets the release of a different category of stem cells, namely stimulating the mobilization of neural stem cells, which follows a different mechanism.

The compounds of the present disclosure can or may be prepared in the form of a prodrug, i e., protected molecular forms that release the compounds of the disclosure after administration to a subject. Typically, protecting groups are hydrolyzed in body fluids, such as in the bloodstream, thus releasing the active compound, or they are oxidized or reduced in vivo to release the active compound A discussion of the design, preparation, and use of prodrugs is found in Smith and Williams Introduction to the Principles of Drug Design, Smith, H. J.; Wright, 2^{nd} ed., London (1988).

The CXCR4-antagonist preferred embodiment compounds of the disclosure, being polyamines, can or may be administered when prepared in the forms of their respective acid addition salts, or metal complexes thereof. Suitable acid addition salts include salts of inorganic acids that are biocompatible, which include HCL, HBr, sulfuric, and phosphoric acids and the like, as well as organic acids such as acetic, propionic, and butyric acid and the like, as well as acids containing more than one carboxyl group, such as oxalic, glutaric, and adipic acid and the like. Typically, at physiological pH, the compounds of the disclosure will be in the forms of their respective acid addition salts. Particularly preferred acid addition salts are the hydrochloride salts. In addition, when prepared as purified forms, the preferred CXCR4-antagonist compounds can or may also be crystallized as their respective hydrates or other pharmaceutically suitable solvates.

The CXCR4-antagonist preferred embodiment compounds of the disclosure can or may be formulated and administered as sole active ingredients, as fixed dose combination admixtures of various compounds of general formula (1), and/or in admixture with additional active ingredients that are therapeutically or nutritionally useful, such as antibiotics, vitamins, herbal extracts, anti-Inflammatories, glucose, antipyretics, analgesics, granulocyte-macrophage colony stimulating factor (GM-CSF), Interleukin-I (IL-1), Interleukin-3 (IL-3), Interleukin-8 (IL-8), PIXY-321 (GM-CSF/IL-3 fusion protein), macrophage inflammatory protein, stem cell factor, thrombopoietin, and the like.

The compounds utilized in the disclosure may be formulated for administration via surgical implantation devices of the present disclosure to a human or an animal subject using commonly understood formulation techniques well known in the art. Formulations which are suitable for particular modes of administration and for compounds of the type represented by those of formula (1) may be found in any edition of Remington's Pharmaceutical Sciences, Mack Publishing Company, Easton, Pa. Once the preferred compounds of the disclosure have been formulated into a pharmaceutically acceptable formulation, then most preferably that formulation is used as a preparation that is utilized to fill or pack the interior space of a nerve bridging conduit, that is designed and selected to restrict the direction of regenerating axonal processes towards the distal nerve segment while at the same time protecting the regenerating axons from intervening scar tissue or trauma The protected microenvironment created by this conduit will typically contain enriched compositions of chemical and biological factors selected to act as neurotrophic factors. Current FDA-approved commercially available conduits in the USA are made of either polyglycolic acid (PGA), collagen, or polycaprolacgone, (PCL). The tubular structure of a neural conduit is most preferably a tube made out of a suitable polymer from among the aformentioned examples that, upon curing, has desired characteristics of length, interior diameter, exterior diameter, wall thickness, elasticity, modulus of elasticity, rigidity, resistance to collapse and kinking, semi-permeability to allow the diffusion of oxygen and nutrients to support nerve regeneration, and absorbability or reabsorb ability, strength, ability to be sterilized, as for example by ethylene oxide (EtO) sterilization, resistance to oxidation that can lead to brittleness or stiffening, shelf life, and compatibility with the tube's contents (both the active component, as well as the other active or inert components of the tube's contents).

In addition to being utilized as therapeutic conduit matrix and filler, preferably, the compounds utilized in the present disclosure are next most desirably administered by injection, inundation, spraying, swabbing, or saturation, in the area of the nerve trauma. The compounds may be administered as a single bolus dose or application, a dose or application over time, or in multiple dosages or applications. Suitable dosage ranges for the compounds of formula (1) vary according to these considerations, but in general, the compounds are adminstered in the range of about 0.1 μg/kg-5 mg/kg of body weight: preferably the range is about 1 μg/kg-300 μg/kg of body weight; and more preferably about 10 μg/kg-100 μg/kg of body weight. For a typical 70-kg human subject, thus, the dosage range is from about 0.7 μg-350 mg; preferably about 700 μg-21 mg; most preferably about 700 μg-7 mg. Subjects that will respond favorably to the method of the disclosure include medical and veterinary subjects generally, including human patients. Among other subjects for whom the methods of the disclosure is useful are cats, dogs, large animals, avians such as chickens, and the like. In additional aspects, the disclosure is directed to the incorporation into neural conduits of CXCR4 antagonist compounds and suitable pharmaceutical compositions, containing a compound of formula (1) for use in effecting neurogeneration, neuroregeneration, and neural repair.

The preferable CXCR4-antagonist active ingredient compounds for use in the disclosure are exemplified by the genus structural formula (1):

Z-linker-Z′ (1)
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
or Z is of the formula

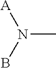

wherein A comprises a monocyclic or bicyclic fused ring system containing at least one N;
and B is H or an organic moiety of 1-20 atoms;
Z′ may be embodied in a form as defined by Z above, or alternatively may be of the formula

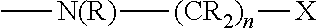

wherein each R is independently H or straight, branched or cyclic alkyl (1-6C),
n is 1 or 2, and
X is an aromatic ring, including heteroaromatic rings, or is a mercaptan;
“linker” represents a bond, alkylene (1-6C) or may comprise aryl, fused aryl, oxygen atoms contained in an alkylene chain, or may contain keto groups or nitrogen or sulfur atoms.

In general, within the group of compounds of formula (1), the preferred embodiments of Z and Z′ are cyclic polyamine moieties having from 9-24C that include 3-5 nitrogen atoms. Particularly preferred are 1,5,9,13-tetraazacyclohexadecane; 1,5,8,11,14-pentaazacyclohexadecane; 1,4,8,11-tetraazacylotetradecane; 1,5,9-triazacyclododecane; 1,4,7,10-tetraazacyclododecane; and the like, including such cyclic polyamines which are fused to an additional aromatic or heteroaromatic rings and/or containing a heteroatom other than nitrogen incorporated in the ring. Embodiments wherein the cyclic polyamine contains a fused additional cyclic system or one or more additional heteroatoms are described in U.S. Pat. Nos. 5,698,546, 7,897,590, and WO 01/44229, the disclosures are incorporated hereinabove by reference.

Also preferred are
3,7,11,17-tetraazabicyclo(13.3.1)heptadeca-1(17), 13,15-triene;
4,7,10,17-tetraazabicyclo(13.3.1)heptadeca-1(17). 13,15-triene;
1,4,7,10-tetraazacyclotetradecane; 1,4,7-triazacyclotetradecane; and
4,7,10-triazabicyclo(13.3.1)heptadeca-1(17), 13,15-triene.

When Z′ is other than a cyclic polyamine as defined in Z, its preferred embodiments are set forth in U.S. Pat. No. 5,817,807, also incorporated herein by reference.

Preferred forms of the formula (1) are where Z is of the formula

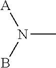

and wherein A comprises a monocyclic or bicycle fused ring system containing at least one N and B is H or an organic moiety of 1-20 atoms, are disclosed in WO 00/56729, WO 02/22600; WO 02/34745; and WO 02/22599 all as previously cited above and the entire disclosures of all of said publications are incorporated herein by reference.

Preferred forms of the linker moiety include those wherein the linker is a bond, or wherein the linker includes an aromatic moiety flanked by alkylene, preferably methylene moieties. Preferred linking groups include the methylene bracketed forms of 1,3-phenylene, 2,6-pyridine, 3,5-pyridine, 2,5-thiophene, 4,4′-(2,2′-bipyrimidine); 2,9-(1,10-phenanthroline) and the like. A particularly preferred linker is 1,4-phenylene-bis-(methylene).

Particularly preferred embodiments of the compound of the formula (1) include 2,2′-bicyclam; 6,6′-bicyclam; the embodiments set forth in U.S. Pat. Nos. 5,021,409, and 6,001,826, and in particular 1,1′-[1,4-phenylene-bis(methylene)]-bis-1,4,8,11-tetraazacyclotetradecane, set forth in U.S. Pat. No. 5,583,131, the entire disclosure of which is incorporated herein by reference, and where said compound is designated herein as AMD3100.

Additional preferred active compounds exhibiting the desired pharmacological activities of being CXCR4 antagonists include:
N-[1,4,8,11-tetraazacyclotetradecanyl-1,4-phenylenebis(methylene)]-2-aminomethyl)pyridine;
7,7′-[1,4-phenylenebis(methylene)]bis-4,7,10,17-tetraazabicyclo-[13.3.1]heptadeca-1(17),13,15-triene;
7,7′-[1,4-phenylenebis(methylene)]bis-3,7,11,17-tetraazabicyclo[13.3.1]heptadeca-1(17), 13,15-trione;
1,1′-[1,3-phenylenebis(methylene)]-bis-1,4,8,11-tetra-azacyclotetradecane;
1,1′-[1,4-phenylenebis(methylene)]-bis-1,4,8,11-tetra-azacyclotetradecane;
1,1′-[1,4-phenylene-bis-(methylene)]-bis-1,4,7,10-tetraazacyclotetradecane;
1,1′-[1,3-phenylene-bis-(methylene)]-bis-1,4,7,10-tetraazacyclotetradecane;
11,11′-(1,2-propanediyl)bis-1,4,8,11-tetraazacyclotetradecane;
N-[4-(1,4,7-triazacyclotetra-decane)-1,4-phenylenebis(methylene)]-2-(aminomethyl)pyridine;
N-[7-(4,7,10-triazabicyclo[13.3.1]heptadeca-1(17), 13,15-triene)-1,4-phenylenebis(methylene)]-2-(aminomethyl)pyridine;
N-[7-(4,7,10,17-tetraazabicyclo[13.3.1]heptadeca-1(17), 13,15-triene)-1,4-phenylenebis(methylene)]-2-(aminomethyl)pyridine.
N-[4-[4,7,10,17-tetraazabicyclo[13.3.1]heptadeca-1(17), 13,15-triene]-1,4-phenylenebis(methylene)]-2-(aminomethyl)pyridine;
3,3′-(bis-1,5,9,13-tetraazacyclohexadecane);
3,3′-(bis-1,5,8,11,14-pentaazacyclohexadecane), methylene (or polymethylene) di-1-N-1,4,8,11-tetraazacyclotetradecane;
3,3′-bis-1,5,9,13,-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
11,11′-(1,2-ethanediyl)bis-1,4,8,11-tetraazacyclotetradecane,
11,11′-(1,2-propanediyl)bis-1,4,8,11-tetraazacyclotetradecane;
11,11′-(1,2-butanediyl)bis-1,4,8,11-tetraazacyclotetradecane;
11,11′-(1,2-pentanediyl)bis-1,4,8,11-tetraazacyclotetradecane;
11,11′-(1,2-hexanediyl)bis-1,4,8,11-tetraazacyclotetradecane;
3,3′-bis-1,5,9,13-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
11,11′-(1,2-ethanediyl)bis-1,4,8,11-tetraazacyclotetradecane;
11,11′-(1,2-propanediyl)bis-1,4,8,11-tetraazacyclotetradecane;
11,11′-(1,2-butanediyl)bis-1,4,8,11-tetraazacyclotetradecane;
11,11′-(1,2-pentanediyl)bis-1,4,8,11-tetraazacyclotetradecane;
11,11′-(1,2-hexanediyl)bis-1,4,8,11-tetraazacyclotetradecane;
1,1′-[1,3-phenylenebis(methylene)]-bis-1,4,8,11-tetra-azacyclotetradecane;
1,1′-[1,4-phenylenebis(methylene)]-bis-1,4,8,11-tetra-azacyclotetradecane;
1,1′-[3,3′-biphenylene-bis-(methylene)]-bis-1,4,8,11-tetraazacyclotetradecane;
11,11′-[1,4-phenylene-bis-(methylene)]-bis-1,4,7,11-tetraazacyclotetradecane;
1,11′-[1,4-phenylene-bis(methylene)]-1,4,8,1 I-tetraazacyclotetradecane;
1,1′-[2,6-pyridine-bis-(methylene)]-bis-1,4,8,11-tetraazacyclotetradecane;
1,1-[3,5-pyridine-bis-(methylene)]-bis-1,4,8,11-tetraazacyclotetradecane;
1,1′-[2,5-thiophene-bis-(methylene)]-bis-1,4,8,11-tetraazacyclotetradecane;
1,1′-[4,4′-(2,2′-bipyridine)-bis-(methylene)]-bis-1,4,8,11-tetraazacyclotetradecane;
1,1′-[2,9-(1,10-phenanthroline)-bis-(methylene)]-bis-1,4,8,11-tetraazacyclotetradecane;
1,1′-[1,3-phenylene-bis-(methylene)]-bis-1,4,7,10-tetraazacyclotetradecane;
1,1′-[1,4-phenylene-bis-(methylene)]-bis-1,4,7,10-tetraazacyclotetradecane;
1,1′-[5-nitro-1,3-phenylenebis(methylene)]bis-1,4,8,11-tetraazacyclotetradecane;
1,1′-[2,4,5,6-tetrachloro-1,3-phenylenebis(methylene)]bis-1,4,8,11-tetraazacyclotetradecane;
1,1′-[2,3,5,6-tetrafluoro-1,4-phenylenebis(methylene)]bis-1,4,8,11-tetraazacyclotetradecane;
1,1′-[1,4-naphthylene-bis-(methylene)]bis-1,4,8,11-tetraazacyclotetradecane;
1,1′-[1,3-phenylenebis-(methylene)]bis-1,5,9-triazacyclododecane;
1,1′-[1,4-phenylene-bis-(methylene)]-1,5,9-triazacyclododecane;
1,1′-[2,5-dimethyl-1,4-phenylenebis-(methylene)]-bis-1,4,8,11-tetraazacyclotetradecane;
1,1′-[2,5-dichloro-1,4-phenylenebis-(methylene)]-bis-1,4,8,11-tetraazacyclotetradecane;
1,1′-[2-bromo-1,4-phenylenebis-(methylene)]-bis-1,4,8,11-tetraazacyclotetradecane;
1,1′-[6-phenyl-2,4-pyridinebis-(methylene)]-bis-1,4,8,11-tetraazacyclotetradecane;
7,7′-[1,4-phenylene-bis(methylene)]bis-3,7,11,17-tetraazabicyclo[13.3.1]heptadeca-1(17), 13,15-triene;
7,7′-[1,4-phenylene-bis(methylene)]bis[15-chloro-3,7,11,17-tetraazabicyclo[13.3.1]heptadeca-1(17), 13,15-triene];
7,7′-[1,4-phenylene-bis(methylene)]bis[15-methoxy-3,7,11,17-tetraazabicyclo[13.3.1]heptadeca-1(17), 13,15-triene];
7,7′-[1,4-phenylene-bis(methylene)]bis-3,7,11,17-tetraazabicyclo[13.3.1]-heptadeca-13,16-triene-1S-one;
7,7′-[1,4-phenylene-bis(methylene)]bis-4,7,10,17-tetraazabicyclo[13.3.1]-heptadeca-1(17),13,15-triene;
8,8′-[1,4-phenylene-bis(methylene)]bis-4,8,12,19-tetraazabicyclo[15.3.1]nonadeca-1(19), 15,17-triene;
6,6′-[1,4-phenylene-bis(methylene)]bis-3,6,9,15-tetraazabicyclo[11.3.1]pentadeca-1(15), 11,13-triene;
6,6′-[1,3-phenylene-bis(methylene)]bis-3,6,9,15-tetraazabicyclo[11.3.1]pentadeca-1(15), 11,13-triene;
17,17′-[1,4-phenylene-bis(methylene)]bis-3,6,14,17,23,24-hexaazatricyclo[17.3.1,18,12]tetracosa-1(23),8,10,12(24), 19,21-hexaene;
N-[1,4,8,11-tetraazacyclotetradecanyl-1,4-phenylenebis(methylene)]-2-(amino-methyl)pyridine;
N-[1,4,8,11-tetraazacyclotetradecanyl-1,4-phenylenebis(methylene)]-N-methyl-2-(aminomethyl)pyridine:
N-[1,4,8,11-tetraazacyclotetradecanyl-1,4-phenylenebis(methylene)]-4-)amino-methyl)pyridine;
N-[1,4,8,11-tetraazacyclotetradecanyl-1,4-phenylenebis(methylene)]-3-(amino-methyl)pyridine;
N-[1,4,8,11-tetraazacyclotetradecanyl-1,4-phenylenebis(methylene)]-(2-amino-methyl-5-methyl)pyrazine;
N-[1,4,8,11-tetraazacyclotetradecanyl-1,4-phenylenebis(methylene)]-2-(amino-ethyl)pyridine;
N-[1,4,8,11-tetraazacyclotetradecanyl-1,4-phenylenebis(methylene)]-2-(amino-methyl)thiophene;
N-[1,4,8,1 l-tetraazacyclotetradecanyl-1,4-phenylenebis(methylene)]-2-(amino-ethyl)mercaptan;
N-[1,4,8,11-tetraazacyclotetradecanyl-1,4-phenylenebis(methylene)]-2-amino-benzylamine;
N-[1,4,8,11-tetraazacyclotetradecanyl-1,4-phenylenebis(methylene)]-4-amino-benzylamine;
N-[1,4,8,11-tetraazacyclotetradecanyl-1,4-phenylenebis(methylene)]-4-(amino-ethyl)imidazole;
N-[1,4,8,11-tetraazacyclotetradecanyl-1,4-phenylenebis(methylene)]-benzylamine;
N-[1,4,8,11-tetraazacyclotetradecanyl-1,4-phenylenebis(methylene)]-purine,
N-[1,4,8,11-tetraazacyclotetradecanyl-1,4-phenylenebis(methylene)]-4-phenylpiperazine;
N-[4-(1,4,7-triazacyclotetra-decanyl)-1,4-phenylenebis(methylene)]-2-(aminomethyl)pyridine;
N-[7-(4,7,10,17-tetraazabicyclo[13.3.1]heptadeca-1(17), 13,15-trienyl)-1,4-phenylenebis(methylene)]-2-(aminomethyl)pyridine;
N-[7-(4,7,10-triazabicyclo[13.3.1]heptadeca-1(17), 13,15-trienyl)-1,4-phenylenebis(methylene)]-2-(aminomethyl)pyridine;
N-[4-[4,7,10-triazabicyclo[13.3.1]heptadeca-1(17), 13,15-trienyl]-1,4-phenylenebis(methylene)]-2-(aminomethyl)pyridine;
N-[1-(1,4,7-triazacyclotetra-decanyl)-1,4-phenylenebis(methylene)]-2-(aminomethyl)pyridine;
N-[4-[4,7,10,17-tetraazabicyclo[13.3.1]heptadeca-1(17), 13,15-trienyl]-1,4-phenylenebis(methylene)]-2-(aminomethyl)pyridine;
N-[3-(3,6,17-triazabicyclo[13.3.1]heptadeca-1(17), 13,15-trienyl)-1,4-phenylenebis(methylene)]-2-(aminomethyl)pyridine;
N-[3-(3,6,17-triazabicyclo[13.3.1]heptadeca-1(17), 13,15-trienyl)-1,3-phenylenebis(methylene)]-2-(aminomethyl)pyridine;
N-[4-(4,7,17-triazabicyclo[13.3.1]heptadeca-1(17), 13,15-trienyl)-1,4-phenylenebis(methylene)]-2-(aminomethyl)pyridine;
N-[7-(4,7,17-triazabicyclo[13.3.1]heptadeca-1(17), 13,15-trienyl)-1,4, phenylenebis(methylene)]-2-(aminomethyl)pyridine;
N-[6-(3,6,9-triazabicyclo[11.3.1]pentadeca-1(15), 11,13-trienyl)-1,3-phenylenebis(methylene)]-2-(aminomethyl)pyridine;
N-[7-(4,10,17-triazabicyclo[13.3.1]heptadeca-1(17), 13,15-trienyl)-1,4-phenylenebis(methylene)]-2-(aminomethyl)pyridine;
N-[4-(1,7-diazacyclotetradecanyl)-1,4-phenylenebis(methylene)]-2-(aminomethyl)pyridine;
N-[7-(4,10-diazabicyclo[13.3.1]heptadeca-1(17), 13,15-trienyl)-1,4-phenylenebis(methylene)]-2-(aminomethyl)pyridine;
N-[4-(11-fluoro-1,4,7-triazacyclotetradecanyl)-1,4-phenylenebis(methylene)]-2-(aminomethyl)pyridine;
N-[4-(11,11-difluoro-1,4,7-triazacyclotetradecanyl)-1,4-phenylenebis(methylene)]-2-(aminomethyl)pyridine:
N-[4-(1,4,7-triazacyclotetradecan-2-one)-yl))-1,4-phenylenebis(methylene)]-2-(aminomethyl)pyridine;
N-[12-(5-oxa-1,9-diazacyclotetradecanyl)-1,4-phenylenebis(methylene)]-2-(aminomethyl)pyridine;
N-[4-(11-oxa-1,7-diazacyclotetradecanyl)-1,4-phenylenebis(methylene)]-2-(aminomethyl)pyridine:
N-[4-(11-thia-1,7-diazacyclotetradecanyl)-1,4-phenylenebis(methylene)]-2-(aminomethyl)pyridine;
N-[4-(11-sulfoxo-1,7-diazacyclotetradecanyl)-1,4-phenylenebis(methylene)]-2-(aminomethyl)pyridine;
N-[4-(11-sulfono-1,7-diazacyclotetradecanyl)-1,4-phenylenebis(methylene)]-2-(aminomethyl)pyridine:
N-[4-(1,4,7-triazacyclotetradecan-3-one)-yl)-1,4-phenylenebis(methylene)]-2-(aminomethyl)pyridine;
N-(2-pyridinylmethyl)-N′-(6,7,8,9-tetrahydro-5H-cyclohepta[b]pyridin-9-yl)-1,4-benzenedimethanamine;
N-(2-pyridinylmethyl)-N′-(5,6,7,8-tetrahydro-8-quinolinyl)-1,4-benzenedimethanamine;
N-(2-pyridinylmethyl)-N′-(6,7-dihydro-5H-cyclopenta[b]pyridin-7-yl)-1,4-benzenedimethanamine,
N-(2-pyridinylmethyl)-N′-(1,2,3,4-tetrahydro-1-naphthalenyl)-1,4-benzenedimethanamine;
N-(2-pyridinylmethyl)-N′-(1-naphthalenyl)-1,4-benzenedimethanamine;
N-(2-pyridinylmethyl)-N′-(8-quinolinyl)-1,4-benzenedimethanamine;
N-(2-pyridinylmethyl)-N′-[2-[(2-pyridinylmethyl)amino]ethyl]-N′-(1-methyl-1,2,3,4-tetrahydro-8-quinolinyl)-1,4-benzene dimethanamine;
N-(2-pyridinylmethyl)-N′-[2-[(1H-imidazol-2-ylmethyl)amino]ethyl]-N′-(1-methyl-1,2,3,4-tetrahydro-8-quinolinyl)-1,4-benzene dimethanamine;
N-(2-pyridinylmethyl)-N′-(1,2,3,4-tetrahydro-8-quinolinyl)-1,4-benzenedimethanamine;
N-(2-pyridinylmethyl)-N′-[2-[(1H-imidazol-2-ylmethyl)amino]ethyl]-N′-(1,2,3,4-tetrahydro-1-naphthalenyl)-1,4-benzene dimethanamine;
N-(2-pyridinylmethyl)-N′-(2-phenyl-5,6,7,8-tetrahydro-8-quinolinyl)-1,4-benzenedimethanamine;
N,N′-bis(2-pyridinylmethyl)-N′-(2-phenyl-5,6,7,8-tetrahydro-8-quinolinyl)-1,4-benzenedimethanamine:
N-(2-pyridinylmethyl)-N′-(5,6,7,8-tetrahydro-S-quinolinyl)-1,4-benzenedimethanamine;
N-(2-pyridinylmethyl)-N′-(1H-imidazol-2-ylmethyl)-N′-(5,6,7,8-tetrahydro-5-quinolinyl)-1,4-benzenedimethanamine:
N-(2-pyridinylmethyl)-N′-(1H-imidazol-2-ylmethyl)-N′-(5,6,7,8-tetrahydro-8-quinolinyl)-1,4-benzenedimethanamine;
N-(2-pyridinylmethyl)-N′-[(2-amino-3-phenyl)propyl]-N′-(5,6,7,8-tetrahydro-8-quinolinyl)-1,4-benzenedimethanamine;
N-(2-pyridinylmethyl)-N′-(1H-imidazol-4-ylmethyl)-N′-(5,6,7,8-tetrahydro-8-quinolinyl)-1,4-benzenedimethanamine;
N-(2-pyridinylmethyl)-N′-(2-quinolinylmethyl)-N′-(5,6,7,8-tetrahydro-8-quinolinyl)˜1,4-benzenedimethanamine;
N-(2-pyridinylmethyl)-N′-(2-(2-naphthoyl)aminoethyl)-N′-(5,6,7,8-tetrahydro-8-quinolinyl)-1,4-benzenedimethanamine;
N-(2-pyridinylmethyl)-N′-[(S)-(2-acetylamino-3-phenyl)propyl]-N′-(5,6,7,8-tetrahydro-8-quinolinyl)-1,4-benzenedimethanamine;
N-(2-pyridinylmethyl)-N′-[(S)-(2-acetylamino-3-phenyl)propyl]-N′-(5,6,7,8-tetrahydro-8-quinolinyl)-1,4-benzenedimethanamine;
N-(2-pyridinylmethyl)-N′-[3-((2-naphthalenylmethyl)amino)propyl]-N′-(5,6,7,8-tetrahydro-8-quinolinyl)-1,4-benzenedimethanamine;
N-(2-pyridinylmethyl)-N′-[2-(S)-pyrollidinylmethyl]-N′-(5,6,7,8-tetrahydro-8-quinolinyl)-1,4-benzenedimethanamine;
N-(2-pyridinylmethyl)-N′-[2-(R)-pyrollidinylmethyl]-N′-(5,6,7,8-tetrahydro-8-quinolinyl)-1,4-benzenedimethanamine;
N-(2-pyridinylmethyl)-N′-[3-pyrazolylmethyl]-N′-(5,6,7,8-tetrahydro-8-quinolinyl)-1,4-benzenedimethanamine;
N-(2-pyridinylmethyl)-N′-[2-pyrrolylmethyl]-N′-(5,6,7,8-tetrahydro-8-quinolinyl)-1,4-benzenedimethanamine:
N-(2-pyridinylmethyl)-N′-[2-thiophenylmethyl]-N′-(5,6,7,8-tetrahydro-8-quinolinyl)-1,4-benzenedimethanamine
N-(2-pyridinylmethyl)-N′-[2-thiazolylmethyl]-N′-(5,6,7,8-tetrahydro-8-quinolinyl)-1,4-benzenedimethanamine;
N-(2-pyridinylmethyl)-N′-[2-furanylmethyl]-N′-(5,6,7,8-tetrahydro-8-quinolinyl)-1,4-benzenedimethanamine:
N-(2-pyridinylmethyl)-N′-[2-[(phenylmethyl)amino]ethyl]-N′-(5,6,7,8-tetrahydro-8-quinolinyl)-1,4-benzenedimethanamine;
N-(2-pyridinylmethyl)-N′-(2-aminoethyl)-N′-(5,6,7,8-tetrahydro-8-quinolinyl)-1,4-benzenedimethanamine;
N-(2-pyridinylmethyl)-N′-3-pyrrolidinyl-N′-(5,6,7,8-tetrahydro-8-quinolinyl)-1,4-benzenedimethanamine
N-(2-pyridinylmethyl)-N′-4-piperidinyl-N′-(5,6,7,8-tetrahydro-8-quinolinyl)-1,4-benzenedimethanamine;
N-(2-pyridinylmethyl)-N′-[2-[(phenyl)amino]ethyl]-N′-(5,6,7,8-tetrahydro-8-quinolinyl)-1,4-benzenedimethanamine;
N-(2-pyridinylmethyl)-N′-(7-methoxy-1,2,3,4-tetrahydro-2-naphthalenyl)-1,4-benzenedimethanamine:
N-(2-pyridinylmethyl)-N′-(6-methoxy-1,2,3,4-tetrahydro-2-naphthalenyl)-1,4-benzenedimethanamine;
N-(2-pyridinylmethyl)-N′-(1-methyl-1,2,3,4-tetrahydro-2-naphthalenyl)-1,4-benzenedimethanamine;
N-(2-pyridinylmethyl)-N′-(7-methoxy-3,4-dihydronaphthalenyl)-1-(aminomethyl)-4-benzamide;
N-(2-pyridinylmethyl)-N′-(6-methoxy-3,4-dihydronaphthalenyl)-1-(aminomethyl)-4-benzamide:
N-(2-pyridinylmethyl)-N′-(1H-imidazol-2-ylmethyl)-N′-(7-methoxy-1,2,3,4-tetrahydro-2-naphtbalenyl)-1,4-benzenedimethanamine;
N-(2-pyridinylmethyl)-N′-(8-hydroxy-1,2,3,4-tetrahydro-2-naphthalenyl)-1,4-benzenedimethanamine;
N-(2-pyridinylmethyl)-N′-(1H-imidazol-2-ylmethyl)-N′-(8-hydroxy-1,2,3,4-tetrahydro-2-naphthalenyl)-1,4-benzenedimethanamine:
N-(2-pyridinylmethyl)-N′-(8-Fluoro-1,2,3,4-tetrahydro-2-naphthalenyl)-1,4-benzenedimethanamine;
N-(2-pyridinylmethyl)-N′-(1H-imidazol-2-ylmethyl)-N′-(8-Fluoro-1,2,3,4-tetrahydro-2-naphthalenyl)-1,4-benzenedimethanamine;
N-(2-pyridinylmethyl)-N′-(5,6,7,8-tetrahydro-7-quinolinyl)-1,4-benzenedimethanamine;
N-(2-pyridinylmethyl)-N′-(1H-imidazol-2-ylmethyl)-N′-(5,6,7,8-tetrahydro-7-quinolinyl)-1,4-benzenedimethanamine;
N-(2-pyridinylmethyl)-N′-[2-[(2-naphthalenylmethyl)amino]ethyl]-N′-(5,6,7,8-tetrahydro-8-quinolinyl)-1,4-benzenedimethanamine;
N-(2-pyridinylmethyl)-N′-[2-(isobutylamino)ethyl]-N′-(5,6,7,8-tetrahydro-8-quinolinyl)˜1,4-benzenedimethanamine;
N-(2-pyridinylmethyl)-N′-[2-[(2-pyridinylmethyl)amino]ethyl]-N′-(5,6,7,8-tetrahydro-8-quinolinyl)-1,4-benzenedimethanamine;
N-(2-pyridinylmethyl)-N′-[2-[(2-furanylmethyl)amino]ethyl]-N′-(5,6,7,8-tetrahydro-8-quinolinyl)-1,4-benzenedimethanamine;
N-(2-pyridinylmethyl)-N′-(2-guanidinoethyl)-N′-(5,6,7,8-tetrahydro-8-quinolinyl)-1,4-benzenedimethanamine;
N-(2-pyridinylmethyl)-N′-[2-[bis-[(2-methoxy)phenylmethyl]amino]ethyl]-N′-(5,6,7,8-tetrahydro-8-quinolinyl)-1,4-benzene dimethanamine;
N-(2-pyridinylmethyl)-N′-[2-[(1H-imidazol-4-ylmethyl)amino]ethyl]-N′-(5,6,7,8-tetrahydro-8-quinolinyl)-1,4-benzene dimethanamine;
N-(2-pyridinylmethyl)-N′-[2-[(1H-imidazol-2-ylmethyl)amino]ethyl]-N′-(5,6,7,8-tetrahydro-8-quinolinyl)-1,4-benzenedimethanamine;
N-(2-pyridinylmethyl)-N′-[2-(phenylureido)ethyl]-N′-(5,6,7,8-tetrahydro-8-quinolinyl)-1,4-benzenedimethanamine,
N-(2-pyridinylmethyl)-N′—[[N″-(n-butyl)carboxamido]methyl]-N′-(5,6,7,8-tetrahydro-8-quinolinyl)-1,4-benzenedimethanamine;
N-(2-pyridinylmethyl)-N′-(carboxamidomethyl)-N′-(5,6,7,8-tetrahydro-8-quinolinyl)-1,4-benzenedimethanamine;
N-(2-pyridinylmethyl)-N′—[(N″-phenyl)carboxamidomethyl]-N′-(5,6,7,8-tetrahydro-8-quinolinyl)-1,4-benzenedimethanamine;
N-(2-pyridinylmethyl)-N′-(carboxymethyl)-N′-(5,6,7,8-tetrahydro-8-quinolinyl)-1,4-benzenedimethanamine:
N-(2-pyridinylmethyl)-N′-(phenylmethyl)-N′-(5,6,7,8-tetrahydro-8-quinolinyl)-1,4-benzenedimethanamine;
N-(2-pyridinylmethyl)-N′-(1H-benzimidazol-2-ylmethyl)-N′-(5,6,7,8-tetrahydro-8-quinolinyl)-1,4-benzenedimethanamine;
N-(2-pyridinylmethyl)-N′-(5,6-dimethyl-| H-benzimidazol-2-ylmethyl)-N′-(5,6,7,8-tetrahydro-8-quinolinyl)-1,4-benzenedimethanamine (hydrobromide salt);
N-(2-pyridinylmethyl)-N′—(S-nitro-1H-benzimidazol-2-ylmethyl)-N′-(5,6,7,8-tetrahydro-8-quinolinyl)-1,4-benzenedimethanamine;
N-(2-pyridinylmethyl)-N′-[(1H)-5-azabenzimidazol-2-ylmethyl]-N′-(5,6,7,8-tetrahydro-8-quinolinyl)-1,4-benzenedimethanamine;
N-(2-pyridinylmethyl)-N-(4-phenyl-1H-imidazol-2-ylmethyl)-N′-(5,6,7,8-tetrahydro-8-quinolinyl)-1,4-benzenedimethanamine;
N-(2-pyridinylmethyl)-N′-[2-(2-pyridinyl)ethyl]-N′-(5,6,7,8-tetrahydro-8-quinolinyl)-1,4-benzenedimethanamine;
N-(2-pyridinylmethyl)-N′-(2-benzoxazolyl)-N′-(5,6,7,8-tetrahydro-8-quinolinyl)-1,4-benzenedimethanamine;
N-(2-pyridinylmethyl)-N′-(trans-2-aminocyclohexyl)-N′-(5,6,7,8-tetrahydro-8-quinolinyl)-1,4-benzenedimethanamine:
N-(2-pyridinylmethyl)-N′-(2-phenylethyl)-N′-(5,6,7,8-tetrahydro-8-quinolinyl)-1,4-benzenedimethanamine;
N-(2-pyridinylmethyl)-N′-(3-phenylpropyl)-N′-(5,6,7,8-tetrahydro-8-quinolinyl)-1,4-benzenedimethanamine;
N-(2-pyridinylmethyl)-N′-(trans-2-aminocyclopentyl)-N′-(5,6,7,8-tetrahydro-8-quinolinyl)-1,4-benzenedimethanamine:
N-[[4-[[(2-pyridinylmethyl)amino]methyl]phenyl]methyl]-N-(5,6,7,8-tetrahydro-8-quinolinyl)-glycinamide;
N-[[4-[[(2-pyridinylmethyl)amino]methyl]phenyl]methyl]-N-(5,6,7,8-tetrahydro-8-quinolinyl)-(L)-alaninamide;
N-[4-[(2-pyridinylmethyl)amino]methyl]phenyl]methyl]-N-(5,6,7,8-tetrahydro-8-quinolinyl)-(L)-aspartamide:
N-[[4-[[(2-pyridinylmethyl)amino]methyl]phenyl]methyl]-N-(5,6,7,8-tetrahydro-8-quinolinyl)-pyrazinamide;
N-[[4-[[(2-pyridinylmethyl)amino]methyl]phenyl]methyl]-N-(5,6,7,8-tetrahydro-8-quinolinyl)-(L)-prolinamide,
N-[[4-[[(2-pyridinylmethyl)amino]methyl]phenyl]methyl]-N-(5,6,7,8-tetrahydro-8-quinolinyl)-(L)-lysinamide:
N-[[4-[[(2-pyridinylmethyl)amino]methyl]phenyl]methyl]-N-(5,6,7,8-tetrahydro-8-quinolinyl)-benzamide;
N-[4-[(2-pyridinylmethyl)amino]methyl]phenyl]methyl]-N-(5,6,7,8-tetrahydro-8-quinolinyl)-picolinamide;
N′-benzyl-N-[[4-[(2-pyridinylmethyl)amino]methyl]phenyl]methyl]-N-(5,6,7,8-tetrahydro-8-quinolinyl)-urea;
N′-phenyl-N-[[4-[[(2-pyridinylmethyl)amino]methyl]phenyl]methyl]-N-(5,6,7,8-tetrahydro-8-quinolinyl)-urea;
N-(6,7,8,9-tetrahydro-5H-cyclohepta[bacteriapyridin-9-yl)-4-[[(2-pyridinylmethyl)amino]methyl]benzamide,
N-(5,6,7,8-tetrahydro-8-quinolinyl)-4-[(2-pyridinylmethyl)amino]methyl]benzamide;
N,N′-bis(2-pyridinylmethyl)-N′-(5,6,7,8-tetrahydro-8-quinolinyl)-1,4-benzenedimethanamine;
N,N′-bis(2-pyridinylmethyl)-N′-(6,7,8,9-tetrahydro-5H-cyclohepta[bacteriapyridin-9-yl)-1,4-benzenedimethanamine;
N,N′-bis(2-pyridinylmethyl)-N′-(6,7-dihydro-5H-cyclopenta[bacteriapyridin-7-yl)-1,4-benzenedimethanamine;
N,N′-bis(2-pyridinylmethyl)-N′-(1,2,3,4-tetrahydro-1-naphthalenyl)-1,4-benzenedimethanamine;
N,N′-bis(2-pyridinylmethyl)-N′-[(5,6,7,8-tetrahydro-8-quinolinyl)methyl]-1,4-benzenedimethanamine:
N,N′-bis(2-pyridinylmethyl)-N′[(6,7-dihydro-5H-cyclopenta[bacteriapyridin-7-yl)methyl]-1,4-benzenedimethanamine;
N-(2-pyridinylmethyl)-N-(2-methoxyethyl)-N′-(5,6,7,8-tetrahydro-8-quinolinyl)-1,4-benzenedimethanamine;
N-(2-pyridinylmethyl)-N-[2-(4-methoxyphenyl)ethyl]-N′-(5,6,7,8-tetrahydro-8-quinolinyl)-1,4-benzenedimethanamine:
N,N′-bis(2-pyridinylmethyl)-1,4-(5,6,7,8-tetrahydro-8-quinolinyl)benzenedimethanamine;
N-[(2,3-dimethoxyphenyl)methyl]-N′-(2-pyridinylmethyl)-N-(5,6,7,8-tetrahydro-8-quinolinyl)-1,4-benzenedimethanamine;
N,N′-bis(2-pyridinylmethyl)-N-[1-(N″-phenyl-N″-methylureido)-4-piperidinyl]-1,3-benzenedimethanamine;
N,N′-bis(2-pyridinylmethyl)-N—[N″-p-toluenesulfonylphenylalanyl)-4-piperidinyl]-1,3-benzenedimethanamine;
N,N′-bis(2-pyridinylmethyl)-N-[1-[3-(2-chlorophenyl)-5-methyl-isoxazol-4-oyl]-4-piperidinyl]-1,3-benzenedimethanamine;
N-[(2-hydroxyphenyl)methyl]-N′-(2-pyridinylmethyl)-N-(6,7,8,9-tetrahydro-5H-cyclohepta[bacteriapyridin-9-yl)-1,4-benzenedimethanamine:
N-[(4-cyanophenyl)methyl]-N′-(2-pyridinylmethyl)-N-(6,7,8,9-tetrahydro-5H-cyclohepta[bacteriapyridin-9-yl)-1,4-benzenedimethanamine;
N-[(4-cyanophenyl)methyl]-N′-(2-pyridinylmethyl)-N-(5,6,7,8-tetrahydro-8-quinolinyl)-1,4-benzenedimethanamine:
N-[(4-acetamidophenyl)methyl]-N′-(2-pyridinylmethyl)-N-(5,6,7,8-tetrahydro-8-quinolinyl)-1,4-benzenedimethanamine.
N-[(4-phenoxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(1-methyl-2-carboxamido)ethyl]-N,N′-bis(2-pyridinylmethyl)-1,3-benzenedimethanamine;
N-[(4-benzyloxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(thiophene-2-yl)methyl]-N′-(2-pyridinylmethyl)-N-(6,7,8,9-tetrahydro-5H-cyclohepta[bacteriapyridin-9-yl)-1,4-benzenedimethanamine;
N-[1-(benzyl)-3-pyrrolidinyl]-N,N′-bis(2-pyridinylmethyl)-1,3-benzenedimethanamine;
N-[[1-methyl-3-(pyrazol-3-yl)]propyl]-N,N′-bis(2-pyridinylmethyl)-1,3-benzenedimethanamine:
N-[1-(phenyl)ethyl]-N,N′-bis(2-pyridinylmethyl)-1,3-benzenedimethanamine;
N-[(3,4-methylenedioxyphenyl)methyl]-N′-(2-pyridinylmethyl)-N-(6,7,8,9-tetrahydro-5H-cyclohepta[b]pyridin-9-yl)-1,4-benzenedimethanamine;
N-[1-benzyl-3-carboxymethyl-4-piperidinyl]-N, N′-bis(2-pyridinylmethyl)-1,3-benzenedimethanamine;
N-[(3,4-methylenedioxyphenyl)methyl]-N′-(2-pyridinylmethyl)-N-(5,6,7,8-tetrahydro-8-quinolinyl)-1,4-benzenedimethanamine;
N-(3-pyridinylmethyl)-N′-(2-pyridinylmethyl)-N-(6,7,8,9-tetrahydro-5H-cyclohepta[b]pyridin-9-yl)-1,4-benzenedimethanamine;
N-[[1-methyl-2-(2-tolyl)carboxamido]ethyl]-N,N′-bis(2-pyridinylmethyl)-1,3-benzenedimethanamine;
N-[(1,5-dimethyl-2-phenyl-3-pyrazolinone-4-yl)methyl]-N′-(2-pyridinylmethyl)-N-(5,6,7,8-tetrahydro-8-quinolinyl)-1,4-benzenedimethanamine;
N-[(4-propoxyphenyl)methyl]-N′-(2-pyridinylmethyl)-N-(6,7,8,9-tetrahydro-5H-cyclohepta[b]pyridin-9-yl)-1,4-benzenedimethanamine;
N-(1-phenyl-3,5-dimethylpyrazolin-4-ylmethyl)-N′-(2-pyridinylmethyl)-N-(5,6,7,8-tetrahydro-8-quinolinyl)-1,4-benzenedimethanamine;
N-[1H-imidazol-4-ylmethyl]-N,N′-bis(2-pyridinylmethyl)-1,3-benzenedimethanamine;
N-[(3-methoxy-4,5-methylenedioxyphenyl)methyl]-N′-(2-pyridinylmethyl)-N-(6,7,8,9-tetrahydro-5H-cyclohepta[b]pyridin-9-yl)-1,4-benzenedimethanamine:
N-[(3-cyanophenyl)methyl]-N′-(2-pyridinylmethyl)-N-(6,7,8,9-tetrahydro-5H-cyclohepta[b]pyridin-9-yl)-1,4-benzenedimethanamine;
N-[(3-cyanophenyl)methyl]-N′-(2-pyridinylmethyl)-N-(5,6,7,8-tetrahydro-8-quinolinyl)-1,4-benzenedimethanamine:
N-(5-ethylthiophene-2-ylmethyl)-N′-(2-pyridinylmethyl)-N-(6,7,8,9-tetrahydro-5H-cyclohepta[b]pyridin-9-yl)-1,4-benzenedimethanamine:
N-(5-ethylthiophene-2-ylmethyl)-N′-(2-pyridinylmethyl)-N-(5,6,7,8-tetrahydro-8-quinolinyl)-1,4-benzenedimethanamine;
N-[(2,6-difluorophenyl)methyl]-N′-(2-pyridinylmethyl)-N-(6,7,8,9-tetrahydro-5H-cyclohepta[b]pyridin-9-yl)-1,4-benzenedimethanamine;
N-[(2,6-difluorophenyl)methyl]-N′-(2-pyridinylmethyl)-N-(5,6,7,8-tetrahydro-8-quinolinyl)-1,4-benzenedimethanamine:
N-[(2-difluoromethoxyphenyl)methyl]-N′-(2-pyridinylmethyl)-N-(6,7,8,9-tetrahydro-5H-cyclohepta[b]pyridin-9-yl)-1,4-benzenedimethanamine;
N-(2-difluoromethoxyphenylmethyl)-N′-(2-pyridinylmethyl)-N-(5,6,7,8-tetrahydro-8-quinolinyl)-1,4-benzenedimethanamine;
N-(1,4-benzodioxan-6-ylmethyl)-N′-(2-pyridinylmethyl)-N-(6,7,8,9-tetrahydro-5H-cyclohepta[b]pyridin-9-yl)-1,4-benzenedimethanamine:
N,N′-bis(2-pyridinylmethyl)-N-[1-(N″-phenyl-N″-methylureido)-4-piperidinyl]-1,4-benzenedimethanamine;
N,N′-bis(2-pyridinylmethyl)-N—[N″-p-toluenesulfonylphenylalanyl)-4-piperidinyl]-1,4-benzenedimethanamine:
N-[1-(3-pyridinecarboxamido)-4-piperidinyl]-N,N′-bis(2-pyridinylmethyl)-1,4-benzenedimethanamine;
N-[1-(cyclopropylcarboxamido)-4-piperidinyl]-N,N′-bis(2-pyridinylmethyl)-1,4-benzenedimethanamine;
N-[1-(1-phenylcyclopropylcarboxamido)-4-piperidinyl]-N,N′-bis(2-pyridinylmethyl)-1,4-benzenedimethanamine:
N-(1,4-benzodioxan-6-ylmethyl)-N′-(2-pyridinylmethyl)-N-(5,6,7,8-tetrahydro-8-quinolinyl)-1,4-benzenedimethanamine;
N-[1-[3-(2-chlorophenyl)-5-methyl-isoxazol-4-carboxamido]-4-piperidinyl]-N,N′-bis(2-pyridinylmethyl)-1,4-benzenedimethanamine;
N-[1-(2-thiomethylpyridine-3-carboxamido)-4-piperidinyl]-N,N′-bis(2-pyridinylmethyl)-1,4-benzenedimethanamine:
N-[(2,4-difluorophenyl)methyl]-N′-(2-pyridinylmethyl)-N-(5,6,7,8-tetrahydro-8-quinolinyl)-1,4-benzenedimethanamine.
N-(1-methylpyrrol-2-ylmethyl)-N′-(2-pyridinylmethyl)-N-(5,6,7,8-tetrahydro-8-quinolinyl)-1,4-benzenedimethanamine;
N-[(2-hydroxyphenyl)methyl]-N′-(2-pyridinylmethyl)-N-(5,6,7,8-tetrahydro-8-quinolinyl)-1,4-benzenedimethanamine:
N-[(3-methoxy-4,5-methylenedioxyphenyl)methyl]-N′-(2-pyridinylmethyl)-N-(5,6,7,8-tetrahydro-8-quinolinyl)-1,4-benzenedimethanamine;
N-(3-pyridinylmethyl)-N′-(2-pyridinylmethyl)-N-(5,6,7,8-tetrahydro-8-quinolinyl)-1,4-benzenedimethanamine;
N-[2-(N″-morpholinomethyl)-1-cyclopentyl]-N,N′-bis(2-pyridinylmethyl)-1,4-benzenedimethanamine:
N-[(1-methyl-3-piperidinyl)propyl]-N,N′-bis(2-pyridinylmethyl)-1,4-benzenedimethanamine;
N-(1-methylbenzimidazol-2-ylmethyl)-N′-(2-pyridinylmethyl)-N-(5,6,7,8-tetrahydro-8-quinolinyl)-1,4-benzenedimethanamine;
N-[1-(benzyl)-3-pyrrolidinyl]-N,N′-bis(2-pyridinylmethyl)-1,4-benzenedimethanamine;
N-[[(1-phenyl-3-(N″-morpholino)]propyl]-N,N′-bis(2-pyridinylmethyl)-1,4-benzenedimethanamine;
N-[1-(iso-propyl)-4-piperidinyl]-N,N′-bis(2-pyridinylmethyl)-1,4-benzenedimethanamine;
N-[1-(ethoxycarbonyl)-4-piperidinyl]-N′-(2-pyridinylmethyl)-N-(5,6,7,8-tetrahydro-8-quinolinyl)-1,4-benzenedimethanamine;
N-[(1-methyl-3-pyrazolyl)propyl]-N′-(2-pyridinylmethyl)-N-(5,6,7,8-tetrahydro-8-quinolinyl)-1,4-benzenedimethanamine;
N-[1-methyl-2-(N″,N″-diethylcarboxamido)ethyl]-N,N′-bis(2-pyridinylmethyl)-1,4-benzenedimethanamine;
N-[(1-methyl-2-phenylsulfonyl)ethyl]-N′-(2-pyridinylmethyl)-N-(5,6,7,8-tetrahydro-8-quinolinyl)-1,4-benzenedimethanamine;
N-[(2-chloro-4,5-methylenedioxyphenyl)methyl]-N′-(2-pyridinylmethyl)-N-(5,6,7,8-tetrahydro-8-quinolinyl)-1,4-benzenedimethanamine;
N-[1-methyl-2-[N″-(4-chlorophenyl)carboxamido]ethyl]-N′-(2-pyridinylmethyl)-N-(5,6,7,8-tetrahydro-8-quinolinyl)-1,4-benzenedimethanamine;
N-(1-acetoxyindol-3-ylmethyl)-N′-(2-pyridinylmethyl)-N-(6,7,8,9-tetrahydro-5H-cyclohepta[b]pyridin-9-yl)-1,4-benzenedimethanamine;
N-[(3-benzyloxy-4-methoxyphenyl)methyl]-N′-(2-pyridinylmethyl)-N-(6,7,8,9-tetrahydro-5H-cyclohepta[b]pyridin-9-yl)-1,4-benzenedimethanamine:
N-(3-quinolylmethyl)-N′-(2-pyridinylmethyl)-N-(5,6,7,8-tetrahydro-8-quinolinyl)-1,4-benzenedimethanamine;
N-[(8-hydroxy)-2-quinolylmethyl]-N′-(2-pyridinylmethyl)-N-(6,7,8,9-tetrahydro-5H-cyclohepta[b]pyridin-9-yl)-1,4-benzenedimethanamine;
N-(2-quinolylmethyl)-N′-(2-pyridinylmethyl)-N-(6,7,8,9-tetrahydro-5H-cyclohepta[b]pyridin-9-yl)-1,4-benzenedimethanamine;
N-[(4-acetamidophenyl)methyl]-N′-(2-pyridinylmethyl)-N-(6,7,8,9-tetrahydro-5H-cyclohepta[b]pyridin-9-yl)-1,4-benzenedimethanamine;
N-[1H-imidazol-2-ylmethyl]-N,N′-bis(2-pyridinylmethyl)-1,4-benzenedimethanamine:
N-(3-quinolylmethyl)-N′-(2-pyridinylmethyl)-N-(6,7,8,9-tetrahydro-5H-cyclohepta[b]pyridin-9-yl)-1,4-benzenetrimethanamine;
N-(2-thiazolylmethyl)-N′-(2-pyridinylmethyl)-N-(6,7,8,9-tetrahydro-5H-cyclohepta[b]pyridin-9-yl)-1,4-benzenedimethanamine;
N-(4-pyridinylmethyl)-N′-(2-pyridinylmethyl)-N-(6,7,8,9-tetrahydro-5H-cyclohepta[b]pyridin-9-yl)-1,4-benzenedimethanamine;
N-[(5-benzyloxy)benzo[b]pyrrol-3-ylmethyl]-N,N′-bis(2-pyridinylmethyl)-1,4-benzenedimethanamine;
N-(1-methylpyrazol-2-ylmethyl)-N′-(2-pyridinylmethyl)-N-(6,7,8,9-tetrahydro-5H-cyclohepta[b]pyridin-9-yl)-1,4-benzenedimethanamine;
N-[(4-methyl)-1H-imidazol-5-ylmethyl]-N,N′-bis(2-pyridinylmethyl)-1,4-benzenedimethanamine;
N-[[(4-dimethylamino)-1-napthalenyl]methyl]-N,N′-bis(2-pyridinylmethyl)-1,4-benzenedimethanamine;
N-[1,5-dimethyl-2-phenyl-3-pyrazolinone-4-ylmethyl]-N,N′-bis(2-pyridinylmethyl)-1,4-benzenedimethanamine:
N-[1-[(1-acetyl-2-(R)-prolinyl]-4-piperidinyl]-N-[2-(2-pyridinyl)ethyl]-N′-(2-pyridinylmethyl)-1,3-benzenedimethanamine;
N-[1-[2-acetamidobenzoyl-4-piperidinyl]-4-piperidinyl]-N-[2-(2-pyridinyl)ethyl]-N′-(2-pyridinylmethyl)-1,3-benzenedimethanamine;
N-[(2-cyano-2-phenyl)ethyl]-N′-(2-pyridinylmethyl)-N-(6,7,8,9-tetrahydro-5H-cyclohepta[b]pyridin-9-yl)-1,4-benzenedimethanamine;
N—[(N″-acetyltryptophanyl)-4-piperidinyl]-N-[2-(2-pyridinyl)ethyl]-N′-(2-pyridinylmethyl)-1,3-benzenedimethanamine;
N—[(N″-benzoylvalinyl)-4-piperidinyl]-N-[2-(2-pyridinyl)ethyl]-N′-(2-pyridinylmethyl)-1,3-benzenedimethanamine;
N-[(4-dimethylaminophenyl)methyl]-N′-(2-pyridinylmethyl)-N-(6,7,8,9-tetrahydro-5H-cyclohepta[b]pyridin-9-yl)-1,4-benzenedimethanamine;
N-(4-pyridinylmethyl)-N′-(2-pyridinylmethyl)-N-(5,6,7,8-tetrahydro-8-quinolinyl)˜1,4-benzenedimethanamine;
N-(1-methylbenzimadazol-2-ylmethyl)-N′-(2-pyridinylmethyl)-N-(6,7,8,9-tetrahydro-5H-cyclohepta[b]pyridin-9-yl)-1,4-benzenedimethanamine;
N-[1-butyl-4-piperidinyl]-N-[2-(2-pyridinyl)ethyl]-N′-(2-pyridinylmethyl)-1,3-benzenedimethanamine:
N-[1-benzoyl-4-piperidinyl]-N-[2-(2-pyridinyl)ethyl]-N′-(2-pyridinylmethyl)-1,3-benzenedimethanamine;
N-[1-(benzyl)-3-pyrrolidinyl]-N-[2-(2-pyridinyl)ethyl]-N′-(2-pyridinylmethyl)-1,3-benzenedimethanamine;
N-[(1-methyl)benzo[b]pyrrol-3-ylmethyl]-N-[2-(2-pyridinyl)ethyl]-N′-(2-pyridinylmethyl)-1,3-benzenedimethanamine:
N-[1H-imidazol-4-ylmethyl]-N-[2-(2-pyridinyl)ethyl]-N′-(2-pyridinylmethyl)-1,3-benzenedimethanamine;
N-[1-(benzyl)-4-piperidinyl]-N-[2-(2-pyridinyl)ethyl]-N′-(2-pyridinylmethyl)-1,4-benzenedimethanamine,
N-[1-methylbenzimidazol-2-ylmethyl]-N-[2-(2-pyridinyl)ethyl]-N′-(2-pyridinylmethyl)-1,4-benzenedimethanamine;
N-[(2-phenyl)benzo[b]pyrrol-3-ylmethyl]-N-[2-(2-pyridinyl)ethyl]-N′-(2-pyridinylmethyl)-1,4-benzenedimethanamine;
N-[(6-methylpyridin-2-yl)methyl]-N′-(2-pyridinylmethyl)-N-(5,6,7,8-tetrahydro-8-quinolinyl)-1,4-benzenedimethanamine:
N-(3-methyl-1H-pyrazol-5-ylmethyl)-N′-(2-pyridinylmethyl)-N-(5,6,7,8-tetrahydro-8-quinolinyl)-1,3-benzenedimethanamine;
N-[(2-methoxyphenyl)methyl]-N′-(2-pyridinylmethyl)-N-(5,6,7,8-tetrahydro-8-quinolinyl)-1,3-benzenedimethanamine:
N-[(2-ethoxyphenyl)methyl]-N′-(2-pyridinylmethyl)-N-(6,7,8,9-tetrahydro-5H-cyclohepta[b]pyridin-9-yl)-1,3-benzenedimethanamine:
N-(benzyloxyethyl)-N′-(2-pyridinylmethyl)-N-(5,6,7,8-tetrahydro-8-quinolinyl)-1,3-benzenedimethanamine;
N-[(2-ethoxy-1-naphthalenyl)methyl]-N′-(2-pyridinylmethyl)-N-(5,6,7,8-tetrahydro-8-quinolinyl)-1,3-benzenedimethanamine;
N-[(6-methylpyridin-2-yl)methyl]-N′-(2-pyridinylmethyl)-N-(5,6,7,8-tetrahydro-8-quinolinyl)-1,3-benzenedimethanamine.
1-[[4-[(2-pyridinylmethyl)amino]methyl]phenyl]methyl]guanidine;
N-(2-pyridinylmethyl)-N-(8-methyl-8-azabicyclo[3.2.1]octan-3-yl)-1,4-benzenedimethanamine;
1-[[4-[[(2-pyridinylmethyl)amino]methyl]phenyl]methyl]homopiperazine;
1-[[3-[(2-pyridinylmethyl)amino]methyl]phenyl]methyl]homopiperazine;
trans and cis-1-[[4-[[(2-pyridinylmethyl)amino]methyl]phenyl]methyl]-3,5-piperidinediamine;
N,N′-[1,4-phenylenebis(methylene)]bis-4-(2-pyrimidyl)piperazine;
1-[[4-[[(2-pyridinylmethyl)amino]methyl]phenyl]methyl]-1-(2-pyridinyl)methylamine;
2-(2-pyridinyl)-5-[[(2-pyridinylmethyl)amino]methyl]-1,2,3,4-tetrahydroisoquinoline:
1-[[4-[[(2-pyridinylmethyl)amino]methyl]phenyl]methyl]-3,4-diaminopyrrolidine;
1-[[4-[(2-pyridinylmethyl)amino]methyl]phenyl]methyl]-3,4-diacetylaminopyrrolidine;
8-[[4-[[(2-pyridinylmethyl)amino]methyl]phenyl]methyl]-2,5,8-triaza-3-oxabicyclo[4.3.0]nonane; and
8-[[4-[[(2-pyridinylmethyl)amino]methyl]phenyl]methyl]-2,5,8-triazabicyclo[4.3.0]nonane.

With no convincing clinical data that one commercially available conduit is better than another, the important parameters to keep in mind when considering conduit repair are nerve diameter and gap size. Regardless of the size of the nerve being repaired, the selected conduit diameter should closely fit the stumps. The use of an undersized conduit is not only conceptually poor given the likely resultant constriction of the regenerating nerve, but also technically difficult for the surgeon to squeeze the stump into a tight conduit.

All of the compositions and methods disclosed and claimed herein can be made and executed by one of ordinary skill in the art without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described or referred to herein without departing from the concept, spirit and scope of the disclosure.

More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

Additionally, the disclosures of PCT patent application PCT/US14/16905, and U.S. Patent Publication No. 20070010831 are incorporated herein by reference as to their complete disclosures and teachings.

While the above description contains much specificity, these should not be construed as limitations on the scope of any embodiment, but as exemplifications of the presented embodiments thereof. Many other alternative embodiments and variations are possible within the teachings of the various embodiments. While the disclosure has been described with reference to exemplary embodiments, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure will not be limited to the particular embodiment disclosed as the best or only mode contemplated for carrying out this disclosure, but that the disclosure will include all embodiments falling within the scope of the appended claims. Also, in the description, there have been disclosed exemplary embodiments of the disclosure and, although specific terms may have been employed, they are, unless otherwise stated, used in a generic and descriptive sense only and not for purposes of limitation, the scope of the disclosure therefore not being so limited. Moreover, the use of the terms first, second, etc. do not denote any order or hierarchy of importance, but rather the terms first, second, etc. are used to distinguish one element from another. Furthermore, the use of the terms a, an, etc. do not denote a limitation of quantity, but rather denote the presence of at least one of the referenced items.

While the disclosure has been described, exemplified, and illustrated in reference to certain preferred embodiments thereof, those skilled in the art will appreciate that various changes, modifications, and substitutions can be made therein without departing from the spirit and scope of the disclosure. It is intended, therefore, that the disclosure be limited only by the scope of the claims which follow, and that such claims be interpreted as broadly as is reasonable.

What is claimed is:

1. A method of increasing peripheral nerve growth, regrowth or regeneration, by bridging across an empty critical gap in at least one nerve in a subject in need thereof, comprising administering to said subject singly, or in combination, a CXCR4 antagonist, a STAT3 activator, and/or an agent that increases nitric oxide content.

2. The method of claim 1, wherein:
(a) said CXCR4 antagonist is selected from the group consisting of Plerixafor, BL-8040, or WZ 811;
(b) said STAT3 activator is selected from the group consisting of colivelin, neuroprotective peptide, ruxolitinib phosphate, a JAK1/JAK2 inhibitor; or IL-6, and
(c) said agent that increases nitric oxide content is selected from the group consisting of (+/−)-S-Nitroso-N-acetylpenicillamine, Molsidomine, 3-Morpholinosydnonimine, Hydroxyguanidine sulfate, Tetrahydrobiopterin (THB) dihydrochloride, S-Nitrosoglutathione (GSNO), Streptozotocin (U-9889), Nicorandil, Dephostatin, DETA NONOate, NOC-12, NOC-18, NOC-5, NOC-7, MAHMA NONOate, PAPA NONOate, Sulfo-NONOate disodium salt, Angeliprimes salt, Diethylamine NONOate, NOR-1, NOR-2, NOR-3, NOR-4, Spermine NONOate, beta-Gal NONOate, BNN3, GEA 3162, GEA 5024, Sodium nitroprusside dihydrate, 10-Nitrooleate, BEC, NO-Indomethacin, Pilotyprimes Acid, SE 175, V-PYRRO/NO, Vinyl-L-NIO Hydrochloride, AMI-1, sodium salt, DAF-FM DA (cell permeable), GEA 5583, N-Acetyl-D,L-penicillamine disulfide, SIN-1 A/gammaCD Complex, 4-Phenyl-3-furoxancarbonitrile, JS-K, Lansoprazole Sulfone N-Oxide, NO-Aspirin 1, Glyco-SNAP-2, N,N-Dicarboxymethyl-N,N-dinitroso-p-phenylenediamine (Disodium Salt), (2S)-(+)-Amino-6-iodoacetamidohexanoic acid, 4AF DA, BEC ammonium salt, DAF-2 DA (cell permeable), DAN-1 EE hydrochloride, DD1, DD2, Diethylamine NONOate/AM, Fructose-SNAP-1, Glyco-SNAP-1, Guanylyl Cyclase, Hydroxyguanidine hemisulfate, N-Cyclopropyl-N′-hydroxyguanidine hydrochloride, NOR-5, PROLI NONOate, S-Nitrosocaptopril, 4-(p-methoxyphenyl)-1,3,2-Oxathiazolylium-5-olate, 4-chloro-4-phenyl-1,3,2-Oxathiozolylium-5-olate, 4-phenyl-1,3,2-Oxathiazolylium-5-olate, 4-trifluoro-4-phenyl-1,3,2-Oxathiazolylium-5-olate, Tricarbonyldichloro-ruthenium (II) dimer, DL-alpha-Difluoromethylornithine hydrochloride, Geranylgeranylacetone, N-Nitrosodiethylamine, L-NMMA (citrate), and 3-(Methylnitrosamino)propionitrile. SIN-1 chloride, L-Arginine, SNAP, L-arginine, nitroglycerin, isosorbide dinitrate, isosorbide mononitrate, or a PDE5 inhibitor.

3. The method of claim 1, wherein said method is a surgical procedure that results in bridging critical gaps ranging in length of from at least 3 cm, up to 6 cm.

4. The method of claim 1, wherein administering comprises administering a CXCR4 antagonist alone.

5. The method of claim 1, wherein administering comprises administering a CXCR4 antagonist with a STAT3 activator.

6. The method of claim 1, wherein administering comprises administering a CXCR4 antagonist with an agent that increases nitric oxide content.

7. The method of claim 1, wherein administering comprises administering a CXCR4 antagonist with a STAT3 activator and an agent that increases nitric oxide content.

8. The method of claim 1, optionally further comprising inserting a physical support structure into the critical gap.

9. The method of claim 8, wherein said physical support structure is composed of a material selected from the group consisting of poly-lactide acid, polyurethane, polydioxanone, silicone, cellulose, collagen, PLGA, polycaprolactone or processed natural extracellular matrix.

10. The method of claim 1, optionally further comprising administering to said subject one or more nerve growth factors.

11. The method of claim 10, wherein said one or more nerve growth factors are selected from the group consisting of
(a) a neurotrophic growth factor selected from the group consisting of nerve growth factor, brain-derived neurotrophic factor, or neurotrophin-3;
(b) a glial-derived growth factor;
(c) a pleotropic nerve growth factor; or
(d) a vascular endothelial growth factor.

12. The method of claim 10, wherein said CXCR4 antagonist, said STAT3 activator, said agent that increases nitric oxide content and/or said one or more nerve growth factors are delivered in a time-dependent release fashion.

13. The method of claim 1, wherein the CXCR4 antagonist is administered prior to both the STAT3 activator or the agent that increases nitric oxide content.

14. The method of claim 1, wherein the CXCR4 antagonist is administered after both the STAT3 activator or the agent that increases nitric oxide content.

15. The method of claim 1, wherein the CXCR4 antagonist is administered between the STAT3 activator and the agent that increases nitric oxide content.

16. The method of claim 1, wherein the subject suffers from a peripheral nervous system deficit.

17. The method of claim 16, wherein said peripheral nerve deficit is congenital.

18. The method of claim 16, wherein said peripheral nerve deficit is due to trauma or an iatrogenic event.

19. The method of claim 16, wherein said peripheral nerve deficit is due to infection.

20. The method of claim 16, wherein said peripheral nerve deficit is due to autoimmune disease.

21. The method of claim 1, wherein said subject suffers from a central nervous system deficit, wherein said central nervous system deficit is a cause of a peripheral nerve deficit.

22. The method of claim 21, wherein said peripheral nerve deficit is caused by a nerve deficit in a cranial nerve or a spinal nerve.

23. The method of claim 22, wherein said spinal nerve deficit is congenital.

24. The method of claim 22, wherein said spinal nerve deficit is due to trauma or an iatrogenic event.

25. The method of claim 22, wherein said spinal nerve deficit is due to infection.

26. The method of claim 22, wherein said spinal nerve deficit is due to autoimmune disease.

27. The method of claim 22, wherein said spinal nerve deficit is a cervical deficit.

28. The method of claim 22, wherein said spinal nerve deficit is a lumbosacral deficit.

29. The method of claim 23, wherein said spinal nerve deficit is a thoracic deficit.

30. The method of claim 1, wherein said subject is a bird, a reptile or a mammal.

31. The method of claim 1, wherein said subject is a human.

32. The method of claim 1, further comprising treating said subject with physical therapy or nerve deficit therapy prior to, at the time of, or following said method.

33. The method of claim 1, wherein administering said method results in improved sensory function in said subject.

34. The method of claim 33, wherein said sensory function is a nociceptive function.

35. The method of claim 33, wherein said sensory function is a mechanoceptive function.

36. The method of claim 1, wherein administering results in improved motor control in said subject.

37. The method of claim 36, wherein motor control is fine motor control.

38. The method of claim 36, wherein motor control is gross motor control.

39. The method of claim 36, wherein motor control is autonomic nerve control.
